(19) United States
(12) Patent Application Publication
(10) Pub. No.: US 2022/0073470 A1
(43) Pub. Date: Mar. 10, 2022

(54) INHIBITORS OF CGAS ACTIVITY AS THERAPEUTIC AGENTS

(71) Applicant: BELLBROOK LABS, LLC, Madison, WI (US)

(72) Inventors: Robert G. Lowery, Madison, WI (US); Meera Kumar, Madison, WI (US); Matthew Boxer, Frederick, MD (US); David Maloney, Frederick, MD (US); Susan Boyd, Cambridge (GB)

(21) Appl. No.: 17/419,837

(22) PCT Filed: Jan. 3, 2020
(86) PCT No.: PCT/US2020/012250

§ 371 Date: Jun. 30, 2021

Related U.S. Application Data
(60) Provisional application No. 62/788,624, filed on Jan. 4, 2019.

(51) Int. Cl. C07D 231/20; C07C 237/48; C07D 401/04; C07D 487/04; C07D 239/94; C07D 213/81; A61P 37/04; C07D 403/04

(52) U.S. Cl. CPC C07D 231/20; C07C 237/48; C07D 401/04; C07D 403/04; C07D 239/94; C07D 213/81; A61P 37/04; C07D 487/04

(57) ABSTRACT

This disclosure relates to compounds, pharmaceutical compositions comprising them, and methods of using the compounds and compositions for treating or preventing inappropriate activation of a type I interferon (IFN) response in a subject in need thereof.

### CROSS-REFERENCE TO RELATED APPLICATIONS

This application claims the benefit of priority of U.S. Provisional Patent Application No. 62/788,624, filed Jan. 4, 2019, all of which is incorporated by reference in its entirety.

### BACKGROUND OF THE INVENTION

### Field of the Disclosure

This disclosure relates to compounds, pharmaceutical compositions comprising them, and methods of using the compounds and compositions for treating or preventing inappropriate activation of a type I interferon (IFN) response in a subject in need thereof.

### Description of Related Art

Cyclic GMP-AMP synthase (cGAS) (UniProtKB—Q8N884) is a recently discovered enzyme that acts as a DNA sensor to elicit an immune response to pathogens via activation of the stimulator of interferon genes (STING) receptor. Shortly after its discovery in 2013, aberrant activation of cGAS by self-DNA was shown to underlie debilitating and sometimes fatal autoimmune diseases, such as systemic lupus erythematosus (SLE), scleroderma, and Aicardi—Goutieres Syndrome (AGS). Knockout studies in animal models have indicated that inhibiting cGAS is a promising approach for therapeutic intervention. Additionally, recent studies have shown that the cGAS-STING pathway plays a key role in the innate immune response to tumors, and stimulation of the pathway is a promising strategy being tested clinically for cancer immunotherapy.

No drugs have been approved specifically for AGS or any other monogenic type I interferonopathies. Current treatment options are limited to intravenous or oral immuno-suppressors and intravenous immunoglobulins during the acute phases, with often only partial control of the flares. Similarly, SLE is treated with over-the counter anti-inflammatories, corticosteroids, and immunosupressives, such as cyclophosphamide and methotrexate, with serious side effects including cancer. The only targeted therapy approved for SLE is BENLYSTA (belimumab), a monoclonal antibody (mAb) against B-cell activating factor (BAFF). BENLYSTA reduces the risk of severe flares and allows lower doses of immunosuppressive in most patients but is not curative.

Accordingly, there remains a need for compounds that can effectively inhibit cGAS activity and treat diseases resulting from aberrant activation of cGAS.

### SUMMARY OF THE DISCLOSURE

The disclosure provides novel inhibitors of cGAS activity. Thus, one aspect of the disclosure provides a compound of formula (I):

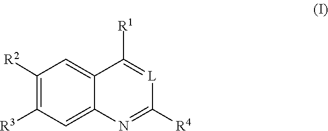

optionally in the form of a pharmaceutically acceptable salt, N-oxide, and/or a solvate or hydrate thereof, wherein:
L is —N— or —CR⁵—;
   R⁵ is hydrogen, halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R^{1C}, —C(O)OR^{1C}, —C(O)NR^{1C}R^{1D}, —S(O)₀₋₂—R^{1C}, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A};
R¹ is aryl optionally substituted with one or more R^{1B}, heteroaryl optionally substituted with one or more R^{1B}, heterocycloalkyl optionally substituted with one or more R^{1A}, C₄-C₈ cycloalkyl optionally substituted with one or more R^{1A}, or —NH(C₁-C₆ alkyl) optionally substituted with one or more R^{1A};
R² is hydrogen, halogen, —NO₂, —CN, 1-C₆ alkyl, 1-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A};
R³ is hydrogen, halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, or C₁-C₆ haloalkoxy; and
R⁴ is hydrogen, halogen, —NO₂, —CN, C₁-C₆ alkyl, C₂-C₈ alkenyl, C₂-C₈ alkynyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R^{1C}, —C(O)OR^{1C}, —C(O)NR^{1C}R^{1D}, —S(O)₀₋₂—R^{1C}, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A};
wherein
   each R^{1A} is independently selected from the group consisting of oxo, halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R^{1C}, —C(O)OR^{1C}, —C(O)NR^{1C}R^{1D}, and —S(O)₀₋₂—R^{1C};
   each R^{1B} is independently selected from the group consisting of halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R^{1C}, —C(O)OR^{1C}, —C(O)NR^{1C}R^{1D}, and —S(O)₀₋₂—R^{1C};
   each R^{1C} is independently selected from the group consisting of hydrogen, C₁-C₆ alkyl, aryl(C₀-C₄ alkyl), heteroaryl(C₀-C₄ alkyl), heterocyclyl(C₀-C₄ alkyl), and cyclyl(C₀-C₄ alkyl); and
   each R^{1D} is independently hydrogen or C₁-C₆ alkyl.

Another aspect of the disclosure provides a compound of formula (II):

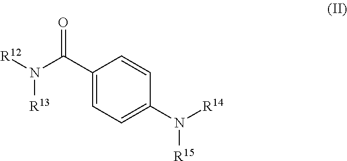

optionally in the form of a pharmaceutically acceptable salt, N-oxide, and/or a solvate or hydrate thereof, wherein:
R¹² is selected from C₁-C₈ alkyl, C₂-C₈ alkenyl and C₂-C₈ alkynyl, each optionally substituted with 1-2 R^{12A}, cycloalkyl and heterocycloalkyl, each optionally substituted with 1-2 R^{12A}, aryl and heteroaryl, each optionally substituted with 1-5 R^{12A}, in which each R^{12A} is independently oxo, optionally substituted C₁-C₄ alkyl, C₁-C₄ fluoroalkyl, halogen, —CN, —SF₅, —N₃, —C(O)OR^{12B}, —SR^{12B}, —S(O)₁₋₂R^{12B}, —OR^{12B}, —(OCH₂CH₂O)ₙ—R^{12C} in which n is 1-4, —N(R^{12C})C(O)CH₂—O—(CH₂CH₂O)ₙ—R^{12C} in which n is 0-3, —C(O)NR^{12B}(CH₂CH₂O)ₙR^{12C} in which n is 0-3, —NR^{12C}R^{12B} and —C(O)R^{12B}, each R^{12B} is independently selected from H, C₁-C₃ alkyl, and —S(O)₁₋₂N(R^{12C})R^{12C}, and each R^{12C} is independently selected from H and C₁-C₃ alkyl,
R¹³ and R¹⁴ are each independently selected from H and C₁-C₃ alkyl; and
R¹⁵ is selected from —S(O)₁₋₂R^{15A} and —C(O)R^{15A};
wherein
   in which R^{15A} is C₁-C₈ alkyl, C₂-C₈ alkenyl and C₂-C₈ alkynyl, each optionally substituted with 1-2 R^{15B}, cycloalkyl and heterocycloalkyl, each optionally substituted with 1-2 R^{15B}, aryl and heteroaryl, each optionally substituted with 1-5 R^{15B},
   in which each R^{15B} is independently halogen, —CN, —NHR^{15C}, —OR^{15C}, C₁-C₈ alkyl, C₂-C₈ alkenyl and C₂-C₈ alkynyl, each optionally substituted with 1-2 R^{15C}, cycloalkyl and heterocycloalkyl, each optionally substituted with 1-2 R^{15C}, aryl and heteroaryl, each optionally substituted with 1-5 R^{15C},
   in which each R^{15C} is independently halogen, C₁-C₄ alkyl, or C₁-C₄ alkoxy.

Another aspect of the disclosure provides pharmaceutical compositions comprising one or more of compounds of the disclosure (e.g., compounds as described above with respect to formula (I) or (II)) and an appropriate carrier, solvent, adjuvant, or diluent.

The disclosure also provides a method for treating or preventing inappropriate activation of a type I interferon (IFN) response in a subject in need thereof, comprising administering to the subject an effective amount of one or more of the compounds of formula (I) or (II), as discussed above.

In embodiment of the methods disclosed herein, the inappropriate activation of a type I IFN response comprises an autoimmune disorder (e.g., Aicardi-Goutieres Syndrome (AGS), retinal vasculopathy with cerebral leukodystropy (RVCL), lupus erythematosus (SLE), scleroderma, or Sjögren's syndrome (SS)). Other aspects of the disclosure will be apparent to the person of ordinary skill in the art in view of the disclosure herein.

Another aspect of the disclosure provides a method of treating an autoimmune disorder, the method comprising administering to a subject in need of such treatment an effective amount of one or more compounds of the disclosure (e.g., compounds as described above with respect to formula (I) or (II)) or pharmaceutical compositions of the disclosure

In certain embodiments of this aspect, the autoimmune disorder is AGS, RVCL, SLE, scleroderma, SS, age-related macular degeneration (AMD), pancreatitis, ischemia (e.g., ischemic injury), inflammatory bowel disease (IBD), nonalcoholic steatohepatitis (NASH), or Parkinson's disease.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 is a schematic showing activation of cGAS by cytoplasmic DNA or RNA initiates activation of the innate immune response via induction of Type I interferons (IFN-I).

FIG. 2 includes A) a schematic showing the cGAS assay principle: enzymatically generated cyclic GAMP (cGAMP) displaces a florescent tracer from mAb causing a decrease in its polarization; B) an image of a coomassie blue stained SDS gel (top) and western blot (bottom) of purified 6×HIS-cGAS (Lane 1) and cGAS-6×His (Lane 2); Z=0.62, Z′=0.7; C) a plot showing detection of purified, full-length human cGAS: cGAS enzyme reactions contained 100 μM ATP and GTP, 62.5 nM 45 bp ISD, 60 min reactions; D) a plot showing the results of screening 3,200 compounds (part of a 100K-compound screen): reaction conditions as in (C): cGAS was used at 10 nM, compounds were at 20 μM; 60 min reaction; negative controls lacked dsDNA (required for cGAS activation); Z=0.62, Z′=0.7; and E) a schematic showing the HTS workflow: primary screen and follow up assays used to triage undesirable compounds and select cGAS inhibitors for advancement into medicinal chemistry/SAR (NSI—non-stoichiometric inhibition, MOA—mechanism of action, SPR—surface plasmon resonance, TSA—thermal shift assay).

FIG. 3 is a schematic of the development of cGAS lead molecules: Iterative rounds of medicinal chemistry informed by biochemical and cellular SAR, structural modeling and ADME/PK testing is used to improve potency, selectivity and CNS efficacy, with a bias toward allosteric inhibitors with long residence times.

FIG. 4 includes a schematic showing the THP1 dual-cell reporter system: secreted luciferase reports on IRF3-driven transcription; secreted alkaline phosphatase reports on NFκB-driven transcription, both downstream of cGAS/STING.

FIG. 5A illustrates activity of IFNβ expression of compound 14 in three different experiments. FIG. 5B illustrates the ISG mRNA expression of compound 14 in THP1-dual cells. Compound 14 in concentration of 200 μM was evaluated after 24 hours.

FIG. 6 illustrates the cytotoxicity evaluation of compound 14 using Cell titer Glo ATP assay. The cells were treated with the test compounds for 24 hours. MnCl₂ used as positive control.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods and compositions described herein can be configured by the person of ordinary skill in the art to meet the desired need. In general, the disclosed materials and methods provide improvements in treatment of diseases or disorders associated with aberrant activation of cGAS. Specifically, the inventors found that the compounds of the disclosure inhibit cGAS activity, and thus can treat or prevent inappropriate activation of a type I IFN response. The compounds of the disclosure are defined generically as with respect to formula (I) or (II), and to various subgenera as defined herein below.

Accordingly, one aspect of the disclosure provides compounds of formula (I):

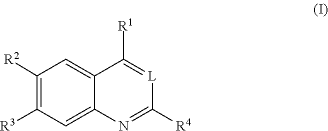

optionally in the form of a pharmaceutically acceptable salt, N-oxide, and/or a solvate or hydrate thereof, wherein L, R¹, R², R³, and R⁴ are as described above.

One embodiment of the disclosure provides compounds of formula (I) as described herein, wherein L is —N—.

Another embodiment of the disclosure provides compounds of formula (I) as described herein, wherein L is —CR⁵— and R⁵ is as described above. In certain embodiments, R⁵ is hydrogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —C(O)OR^{1C}, —C(O)NR^{1C}R^{1D}, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A}. In certain embodiments, R⁵ is hydrogen, C₁-C₆ alkyl, —C(O)OR^{1C}, aryl optionally substituted with one or more R^{1B}, or heteroaryl optionally substituted with one or more R^{1B}. In certain embodiments, R⁵ is hydrogen, phenyl, or —C(O)OH. In certain particular embodiments, R⁵ is hydrogen.

One embodiment of the disclosure provides compounds of formula (I) as described herein, wherein R¹ is heteroaryl optionally substituted with one or more R^{1B}. In certain embodiments, R¹ is imidazol-1-yl optionally substituted with one or more R^{1B}. In certain other embodiments, R¹ is unsubstituted imidazol-1-yl.

Another embodiment of the disclosure provides compounds of formula (I) as described herein, wherein R² is hydrogen, halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or phenyl. In certain embodiments, compounds of formula (I) as described herein are wherein R² is hydrogen, halogen, C₁-C₆ alkyl, —NH₂, C₁-C₆ alkoxy, or phenyl. In certain embodiments, compounds of formula (I) as described herein are wherein R² is hydrogen, methyl, or methoxy. In certain embodiments, compounds of formula (I) as described herein are wherein R² is hydrogen.

One embodiment of the disclosure provides compounds of formula (I) as described herein, wherein R³ is hydrogen, halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy. In certain embodiments, compounds of formula (I) as described herein are wherein R³ is hydrogen, halogen, —NH₂, or methoxy. In certain particular embodiments, compounds of formula (I) as described herein are wherein R³ is hydrogen, —F, —Cl, or —Br. In certain particular embodiments, compounds of formula (I) as described herein are wherein R³ is —Cl.

One embodiment of the disclosure provides compounds of formula (I) as described herein, wherein R⁴ is hydrogen, C₁-C₆ alkyl, —NH₂, —NH(C₁-C₆ alkyl), —N(C₁-C₆ alkyl)₂, aryl optionally substituted with one or more R^{1B}, or heteroaryl optionally substituted with one or more R^{1B}. In certain embodiments, R⁴ is hydrogen, methyl, —NH₂, or phenyl. In certain embodiments, R⁴ is hydrogen, methyl, or phenyl. In certain particular embodiments, compounds of formula (I) as described herein are wherein R⁴ is hydrogen.

In particular embodiments, the compounds of formula (I) as otherwise described herein are wherein R¹ is unsubstituted imidazol-1-yl; R³ is —Cl; and R⁴ is hydrogen, methyl, —NH₂, or phenyl.

In particular embodiments, the compounds of formula (I) as otherwise described herein are wherein R¹ is unsubstituted imidazol-1-yl; R³ is —Cl; and R⁴ is hydrogen.

In certain embodiments, compounds of formula (I) as otherwise described herein are one of compounds of Example 2.

Another aspect of the disclosure provides compounds of formula (II):

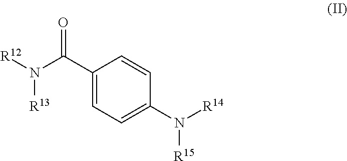

optionally in the form of a pharmaceutically acceptable salt, N-oxide, and/or a solvate or hydrate thereof, wherein R¹², R¹³, R¹⁴, and R¹⁵ are as provided above.

In certain embodiments, compounds of formula (II) as otherwise described herein are those wherein R¹² is aryl or heteroaryl. For example, in certain such embodiments, R¹² is unsubstituted heteroaryl (e.g., 3-pyridinyl). In another example, in certain embodiments as otherwise described herein, R¹² is aryl substituted with 1 R^{12A}. In certain such embodiments, R^{12A} is —C(O)OR^{12B} (e.g., in which R^{12B} is —H).

In certain embodiments of the compounds of formula (II) as otherwise described herein, each of R¹³ and R¹⁴ are —H.

In certain embodiments of the compounds of formula (II) as otherwise described herein, R¹⁵ is —SO₁₋₂R^{15A}. For example, in certain embodiments as otherwise described herein, R¹⁵ is —SO₂R^{15A} in which R^{15A} is aryl substituted with 1-2 R^{15B} (e.g., in which R^{15B} is unsubstituted C₁-C₈ alkyl). In certain embodiments as otherwise described herein, R¹⁵ is —C(O)R^{15A}. For example, in certain embodiments as otherwise described herein, R¹⁵ is —C(O)R^{15A} in which R^{15A} is C₁-C₈ alkyl (e.g., C₁-C₄ alkyl) substituted with 1-2 R^{15B}. In certain such embodiments, R^{15B} is unsubstituted aryl. In other such embodiments, R^{15B} is aryl substituted with 1-2 R^{15C} (e.g., in which R^{15C} is C₁-C₄ alkoxy). In another example, in certain embodiments as otherwise described herein, R¹⁵ is —C(O)R^{15A} in which R^{15A} is aryl substituted with 1-2 R^{15B} (e.g., in which R^{15B} is halogen). In certain such embodiments, each R^{15B} is independently —Br or —I.

In certain embodiments, compounds of formula (II) as otherwise described herein are one of compounds of Example 3.

In certain embodiments, disclosure also provides a cGAS inhibitor compound (e.g., a compound of formula (I) or (II) as discussed above) having an IC₅₀ in the presence of Mn²⁺ that is at least 5-fold less than the IC₅₀ of the compound in otherwise identical conditions but lacking Mn²⁺.

In one embodiment of the disclosure, the compound as otherwise disclosed herein (e.g., a compound of formula (I) or (II), or recited in Example 2 or 3) is in the form of an N-oxide.

In one embodiment of the disclosure, the compound as otherwise disclosed herein (e.g., a compound of formula (I) or (II), or recited in Example 2 or 3) is in the form of a pharmaceutically acceptable salt. The person of ordinary skill in the art will appreciate that a variety of pharmaceutically-acceptable salts may be provided, as described in additional detail below. The person of ordinary skill in the art will appreciate that the phrase “optionally in the form of a pharmaceutically acceptable salt or N-oxide, or a solvate or hydrate” includes compounds in the form of a pharmaceutically acceptable salt of an N-oxide. But in certain embodiments as described above, the compound is not in the form of a pharmaceutically acceptable salt. Thus, in one embodiment, the compound as otherwise disclosed herein is in the form of the base compound.

In one embodiment of the disclosure, the compound as otherwise disclosed herein (e.g., a compound of formula (I) or (II), or recited in Example 2 or 3) is in the form of solvate or hydrate. The person of ordinary skill in the art will appreciate that a variety of solvates and/or hydrates may be formed. The person of ordinary skill in the art will appreciate that the phrase “optionally in the form of a pharmaceutically acceptable salt or N-oxide, or a solvate or hydrate” includes compounds in the form of solvates and hydrates of base compounds, pharmaceutically acceptable salts and N-oxides as described above. But in certain embodiments as described above, the compound is not in the form of a solvate or hydrate.

In one embodiment of the disclosure, the compound as otherwise disclosed herein (e.g., a compound of formula (I) or (II), or recited in Example 2 or 3) is in the form of an N-oxide. But in certain embodiments as described above, the compound is not in the form of an N-oxide.

### Therapeutics Applications

The inventors have determined that, in certain embodiments, the presently described compounds can inhibit cGAS. Accordingly, one aspect of the disclosure provides a method for treating or preventing inappropriate activation of a type I interferon (IFN) response in a subject in need thereof, the method comprising administering to the subject an effective amount of one or more compounds of the disclosure as described herein (e.g., a compound of formula (I) or (II), or those provided in Example 2 or 3) or a pharmaceutical composition of the disclosure as described herein. In certain embodiments of the methods as otherwise described herein, the inappropriate activation of a type I IFN comprises an autoimmune disorder. In certain such embodiments, the autoimmune disorder is Aicardi-Goutieres Syndrome, retinal vasculopathy with cerebral leukodystropy, lupus erythematosus, scleroderma, or Sjögren's syndrome.

The disclosure also provides methods of treating an autoimmune disorder. Such method includes administering to a subject in need of such treatment an effective amount of one or more compounds of the disclosure as described herein or a pharmaceutical composition of the disclosure as described herein.

Many different autoimmune disorders can be treated with compounds and compositions of the disclosure. Autoimmune disorder particularly suitable to be treated by the methods of the disclosure include, but are not limited to, Aicardi-Goutieres Syndrome, retinal vasculopathy with cerebral leukodystropy, lupus erythematosus, scleroderma, and Sjögren's syndrome.

The compounds and compositions of the disclosure as described herein may also be administered in combination with one or more secondary therapeutic agents. Thus, in certain embodiment, the method also includes administering to a subject in need of such treatment an effective amount of one or more compounds of the disclosure as described herein (e.g., a compound of formula (I) or (II), or those provided in Example 2 or 3) or a pharmaceutical composition of the disclosure as described herein and one or more secondary therapeutic agents.

“Combination therapy,” in defining use of a compound of the present disclosure and another therapeutic agent, is intended to embrace administration of each agent in a sequential manner in a regimen that will provide beneficial effects of the drug combination (e.g., the compounds and compositions of the disclosure as described herein and the secondary therapeutic agents can be formulated as separate compositions that are given sequentially), and is intended as well to embrace co-administration of these agents in a substantially simultaneous manner, such as in a single capsule having a fixed ratio of these active agents or in multiple or a separate capsules for each agent. The disclosure is not limited in the sequence of administration: the compounds of and compositions of the disclosure may be administered either prior to or after (i.e., sequentially), or at the same time (i.e., simultaneously) as administration of the secondary therapeutic agent.

In certain embodiments, the secondary therapeutic agent may be administered in an amount below its established half maximal inhibitory concentration (IC₅₀). For example, the secondary therapeutic agent may be administered in an amount less than 1% of, e.g., less than 10%, or less than 25%, or less than 50%, or less than 75%, or even less than 90% of the inhibitory concentration (IC₅₀).

### Pharmaceutical Compositions

In another aspect, the present disclosure provides compositions comprising one or more of compounds as described above with respect to formula (I) or (II), and an appropriate carrier, solvent, adjuvant, or diluent. The exact nature of the carrier, solvent, adjuvant, or diluent will depend upon the desired use for the composition, and may range from being suitable or acceptable for veterinary uses to being suitable or acceptable for human use.

The compounds of the disclosure can be administered, for example, orally, topically, parenterally, by inhalation or spray or rectally in dosage unit formulations containing one or more pharmaceutically acceptable carriers, diluents or excipients. The term parenteral as used herein includes percutaneous, subcutaneous, intravascular (e.g., intravenous), intramuscular, or intrathecal injection or infusion techniques and the like. A medicament including a compound of the disclosure can be provided in any appropriate of the formulations and dosage forms as described herein.

Pharmaceutical compositions can be made using the presently disclosed compounds. For example, in one embodiment, a pharmaceutical composition includes a pharmaceutically acceptable carrier, diluent or excipient, and compound as described above with reference to any one of structural formulae.

In the pharmaceutical compositions disclosed herein, one or more compounds of the disclosure may be present in association with one or more pharmaceutically acceptable carriers, diluents or excipients, and, if desired, other active ingredients. The pharmaceutical compositions containing compounds of the disclosure may be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any suitable method for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by suitable techniques to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Formulations for oral use can also be presented as lozenges.

Aqueous suspensions contain the active materials in admixture with excipients suitable for the manufacture of aqueous suspensions. Such excipients can be suspending agents, for example sodium carboxymethylcellulose, methylcellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

In some embodiments, the pharmaceutically acceptable carrier, diluent, or excipient is not water. In other embodiments, the water comprises less than 50% of the composition. In some embodiments, compositions comprising less than 50% water have at least 1%, 2%, 3%, 4% or 5% water. In other embodiments, the water content is present in the composition in a trace amount.

In some embodiments, the pharmaceutically acceptable carrier, diluent, or excipient is not alcohol. In other embodiments, the alcohol comprises less than 50% of the composition. In some embodiments, compositions comprising less than 50% alcohol have at least 1%, 2%, 3%, 4% or 5% alcohol. In other embodiments, the alcohol content is present in the composition in a trace amount.

Syrups and elixirs can be formulated with sweetening agents, for example glycerol, propylene glycol, sorbitol, glucose or sucrose. Such formulations can also contain a demulcent, a preservative, flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils can be employed as a solvent or suspending medium. For this purpose any bland fixed oil can be employed including synthetic mono- or diglycerides. In addition, fatty acids such as oleic acid find use in the preparation of injectables.

Compounds of the disclosure can also be administered in the form of suppositories, e.g., for rectal administration of the drug. These compositions can be prepared by mixing the compound with a suitable non-irritating excipient that is solid at ordinary temperatures but liquid at the rectal temperature and will therefore melt in the rectum to release the drug. Such materials include cocoa butter and polyethylene glycols.

Compounds of the disclosure can also be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The compositions can be formulated in a unit dosage form of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound described herein. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, 0.1 to about 500 mg of the active ingredient of a compound described herein.

The tablets or pills can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds can vary according to, for example, the particular use for which the treatment is made, the manner of administration of the compound, the health and condition of the patient, and the judgment of the prescribing physician. The proportion or concentration of a compound described herein in a pharmaceutical composition can vary depending upon a number of factors including dosage, chemical characteristics (e.g., hydrophobicity), and the route of administration. For example, the compounds described herein can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the nd extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds described herein can also be formulated in combination with one or more additional active ingredients which can include any pharmaceutical agent such as anti-viral agents, vaccines, antibodies, immune enhancers, immune suppressants, anti-inflammatory agents and the like.

### Definitions

The following terms and expressions used herein have the indicated meanings.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Terms used herein may be preceded and/or followed by a single dash, “-”, or a double dash, “=”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and -alkylaryl indicate the same functionality.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety can refer to a monovalent radical (e.g. CH₃—CH₂—), in some circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, e.g., an N-oxide or tetrasubstituted ammonium salt. On occasion a moiety may be defined, for example, as —B-(A)ₐ, wherein a is 0 or 1. In such instances, when a is 0 the moiety is —B and when a is 1 the moiety is —B-A.

As used herein, the term “alkyl” includes a saturated hydrocarbon having a designed number of carbon atoms, such as 1 to 10 carbons (i.e., inclusive of 1 and 10), 1 to 8 carbons, 1 to 6 carbons, 1 to 3 carbons, or 1, 2, 3, 4, 5 or 6. Alkyl group may be straight or branched and depending on context, may be a monovalent radical or a divalent radical (i.e., an alkylene group). For example, the moiety “—(C₁-C₆alkyl)-O—” signifies connection of an oxygen through an alkylene bridge having from 1 to 6 carbons and C₁-C₃ alkyl represents methyl, ethyl, and propyl moieties. Examples of “alkyl” include, for example, methyl, ethyl, propyl, isopropyl, butyl, iso-, sec- and tert-butyl, pentyl, and hexyl.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “alkenyl” as used herein, unsaturated hydrocarbon containing from 2 to 10 carbons (i.e., inclusive of 2 and 10), 2 to 8 carbons, 2 to 6 carbons, or 2, 3, 4, 5 or 6, unless otherwise specified, and containing at least one carbon-carbon double bond. Alkenyl group may be straight or branched and depending on context, may be a monovalent radical or a divalent radical (i.e., an alkenylene group). For example, the moiety “—(C₂-C₆ alkenyl)-O—” signifies connection of an oxygen through an alkenylene bridge having from 2 to 6 carbons. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkynyl” as used herein, unsaturated hydrocarbon containing from 2 to 10 carbons (i.e., inclusive of 2 and 10), 2 to 8 carbons, 2 to 6 carbons, or 2, 3, 4, 5 or 6 unless otherwise specified, and containing at least one carbon-carbon triple bond. Alkynyl group may be straight or branched and depending on context, may be a monovalent radical or a divalent radical (i.e., an alkynylene group). For example, the moiety “—(C₂-C₆ alkynyl)-O—” signifies connection of an oxygen through an alkynylene bridge having from 2 to 6 carbons. Representative examples of alkynyl include, but are not limited to, acetylenyl, 1-propynyl, 2-propynyl, 3-butynyl, 2-pentynyl, and 1-butynyl.

The term “aryl” represents an aromatic ring system having a single ring (e.g., phenyl) which is optionally fused to other aromatic hydrocarbon rings or non-aromatic hydrocarbon or heterocycle rings. “Aryl” includes ring systems having multiple condensed rings and in which at least one is carbocyclic and aromatic, (e.g., 1,2,3,4-tetrahydronaphthyl, naphthyl). Examples of aryl groups include phenyl, 1-naphthyl, 2-naphthyl, indanyl, indenyl, dihydronaphthyl, fluorenyl, tetralinyl, and 6,7,8,9-tetrahydro-5H-benzo[a]cycloheptenyl. “Aryl” also includes ring systems having a first carbocyclic, aromatic ring fused to a nonaromatic heterocycle, for example, 1H-2,3-dihydrobenzofuranyl and tetrahydroisoquinolinyl. The aryl groups herein are unsubstituted or, when specified as “optionally substituted”, can unless stated otherwise be substituted in one or more substitutable positions with various groups as indicated.

The terms “halogen” or “halo” indicate fluorine, chlorine, bromine, and iodine. In certain embodiments of each and every embodiment as otherwise described herein, the term “halogen” or “halo” refers to fluorine or chlorine. In certain embodiments of each and every embodiment described herein, the term “halogen” or “halo” refers to fluorine. The term “fluoroalkyl” indicates an alkyl group (i.e., as otherwise described herein) that is substituted with at least one fluorine. “Fluoroalkyl” includes alkyl groups substituted with multiple fluorines, such as perfluoroalkyl groups. Examples of fluoroalkyl groups include fluoromethyl, difluoromethyl, trifluoromethyl, pentafluoroethyl, 2,2,2-trifluoroethyl, 1,1,1,3,3,3-hexafluoroprop-2-yl and 2,2,3,3,3-pentafluoroprop-1-yl.

The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl may be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl-N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl-N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “heterocycloalkyl” refers to a non-aromatic ring or ring system containing at least one heteroatom that is preferably selected from nitrogen, oxygen and sulfur, wherein said heteroatom is in a non-aromatic ring. The heterocycloalkyl may have 1, 2, 3 or 4 heteroatoms. The heterocycloalkyl may be saturated (i.e., a heterocycloalkyl) or partially unsaturated (i.e., a heterocycloalkenyl). Heterocycloalkyl includes monocyclic groups of three to eight annular atoms as well as bicyclic and polycyclic ring systems, including bridged and fused systems, wherein each ring includes three to eight annular atoms. The heterocycloalkyl ring is optionally fused to other heterocycloalkyl rings and/or non-aromatic hydrocarbon rings. In certain embodiments, the heterocycloalkyl groups have from 3 to 7 members in a single ring. In other embodiments, heterocycloalkyl groups have 5 or 6 members in a single ring. In some embodiments, the heterocycloalkyl groups have 3, 4, 5, 6 or 7 members in a single ring. Examples of heterocycloalkyl groups include, for example, azabicyclo[2.2.2]octyl (in each case also “quinuclidinyl” or a quinuclidine derivative), azabicyclo[3.2.1]octyl, 2,5-diazabicyclo[2.2.1]heptyl, morpholinyl, thiomorpholinyl, thiomorpholinyl S-oxide, thiomorpholinyl S,S-dioxide, 2-oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2(1H)-yl, isoindolindionyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S-oxide, tetrahydrothienyl S,S-dioxide and homothiomorpholinyl S-oxide. Especially desirable heterocycloalkyl groups include morpholinyl, 3,4-dihydroisoquinolin-2(1H)-yl, tetrahydropyranyl, piperidinyl, aza-bicyclo[2.2.2]octyl, γ-butyrolactonyl (i.e., an oxo-substituted tetrahydrofuranyl), γ-butryolactamyl (i.e., an oxo-substituted pyrrolidine), pyrrolidinyl, piperazinyl, azepanyl, azetidinyl, thiomorpholinyl, thiomorpholinyl S,S-dioxide, 2-oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as indicated.

The term “cycloalkyl” refers to a non-aromatic carbocyclic ring or ring system, which may be saturated (i.e., a cycloalkyl) or partially unsaturated (i.e., a cycloalkenyl). The cycloalkyl ring optionally fused to or otherwise attached (e.g., bridged systems) to other cycloalkyl rings. Certain examples of cycloalkyl groups present in the disclosed compounds have from 3 to 7 members in a single ring, such as having 5 or 6 members in a single ring. In some embodiments, the cycloalkyl groups have 3, 4, 5, 6 or 7 members in a single ring. Examples of cycloalkyl groups include, for example, cyclohexyl, cyclopentyl, cyclobutyl, cyclopropyl, tetrahydronaphthyl and bicyclo[2.2.1]heptane. The cycloalkyl groups herein are unsubstituted or, when specified as “optionally substituted”, may be substituted in one or more substitutable positions with various groups, as indicated.

The term “ring system” encompasses monocycles, as well as fused and/or bridged polycycles.

The term “oxo” means a doubly bonded oxygen, sometimes designated as ═O or for example in describing a carbonyl “C(O)” may be used to show an oxo substituted carbon.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “substituted,” when used to modify a specified group or radical, means that one or more hydrogen atoms of the specified group or radical are each, independently of one another, replaced with the same or different substituent groups as defined below, unless specified otherwise.

As used herein, the phrase “pharmaceutically acceptable salt” refers to both pharmaceutically acceptable acid and base addition salts and solvates. Such pharmaceutically acceptable salts include salts of acids such as hydrochloric, phosphoric, hydrobromic, sulfuric, sulfinic, formic, toluenesulfonic, methanesulfonic, nitric, benzoic, citric, tartaric, maleic, hydroiodic, alkanoic such as acetic, HOOC—(CH₂)ₙ—COOH where n is 0-4, and the like. Non-toxic pharmaceutical base addition salts include salts of bases such as sodium, potassium, calcium, ammonium, and the like. Those skilled in the art will recognize a wide variety of non-toxic pharmaceutically acceptable addition salts.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. Both the R and the S stereochemical isomers, as well as all mixtures thereof, are included within the scope of the disclosure.

One of ordinary skill in the art of medicinal chemistry also will appreciate that the disclosed structures are intended to include isotopically enriched forms of the present compounds. As used herein “isotopes” includes those atoms having the same atomic number but different mass numbers. As is known to those of skill in the art, certain atoms, such as hydrogen occur in different isotopic forms. For example, hydrogen includes three isotopic forms, protium, deuterium and tritium. As will be apparent to those of skill in the art upon consideration of the present compounds, certain compounds can be enriched at a given position with a particular isotope of the atom at that position. For example, compounds having a fluorine atom, may be synthesized in a form enriched in the radioactive fluorine isotope ¹⁸F. Similarly, compounds may be enriched in the heavy isotopes of hydrogen: deuterium and tritium; and similarly can be enriched in a radioactive isotope of carbon, such as ¹³C. Such isotopic variant compounds undergo different metabolic pathways and can be useful, for example, in studying the ubiquitination pathway and its role in disease. Of course, in certain embodiments, the compound has substantially the same isotopic character as naturally-occurring materials.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the terms “individual,” “patient,” or “subject” are used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” or “effective amount” refers to the amount of active compound or pharmaceutical agent that elicits the biological or medicinal response that is being sought in a tissue, system, animal, individual or human by a researcher, veterinarian, medical doctor or other clinician.

In certain embodiments, an effective amount can be an amount suitable for
(i) inhibiting the progression the disease;
(ii) prophylactic use for example, preventing or limiting development of a disease, condition or disorder in an individual who may be predisposed or otherwise at risk to the disease, condition or disorder but does not yet experience or display the pathology or symptomatology of the disease;
(iii) inhibiting the disease; for example, inhibiting a disease, condition or disorder in an individual who is experiencing or displaying the pathology or symptomatology of the disease, condition or disorder;
(iv) ameliorating the referenced disease state, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease; or
(v) eliciting the referenced biological effect.

As used here, the terms “treatment” and “treating” means (i) ameliorating the referenced disease state, condition, or disorder (or a symptom thereof), such as, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease or symptom thereof, or inhibiting the progression of disease; or (ii) eliciting the referenced biological effect (e.g., inducing apoptosis, or inhibiting glutathione synthesis).

### Methods of Preparation

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as HPLC, preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed. E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry,” Plenum Press, London and New York 1973, in T. W. Greene and P. G. M. Wuts, “Protective Groups in Organic Synthesis,” Third edition, Wiley, New York 1999, in “The Peptides”; Volume 3 (editors: E. Gross and J. Meienhofer), Academic Press, London and New York 1981, in “Methoden der organischen Chemie,” Houben-Weyl, 4.sup.th edition, Vol. 15/I, Georg Thieme Verlag, Stuttgart 1974, in H.-D. Jakubke and H. Jescheit, “Aminosauren, Peptide, Proteine,” Verlag Chemie, Weinheim, Deerfield Beach, and Basel 1982, and/or in Jochen Lehmann, “Chemie der Kohlenhydrate: Monosaccharide and Derivate,” Georg Thieme Verlag, Stuttgart 1974. The protecting groups may be removed at a convenient subsequent stage using methods known from the art.

The compounds disclosed herein can be made using procedures familiar to the person of ordinary skill in the art. For example, the compounds of structural formula (I) or (II) can be prepared according to general procedures of the Examples and/or analogous synthetic procedures. One of skill in the art can adapt the reaction sequences of these Examples and general procedures to fit the desired target molecule. Of course, in certain situations one of skill in the art will use different reagents to affect one or more of the individual steps or to use protected versions of certain of the substituents. Additionally, one skilled in the art would recognize that compounds of the disclosure can be synthesized using different routes altogether.

### EXAMPLES

The compounds and the methods of the disclosure is illustrated further by the following examples, which are not to be construed as limiting the disclosure in scope or spirit to the specific procedures and compounds described in them.

### Example 1. Preparation of Compounds of Formula (I)

The compounds according to formula (I) were prepared essentially according to the following procedures:

1) Preparation of Compound 20

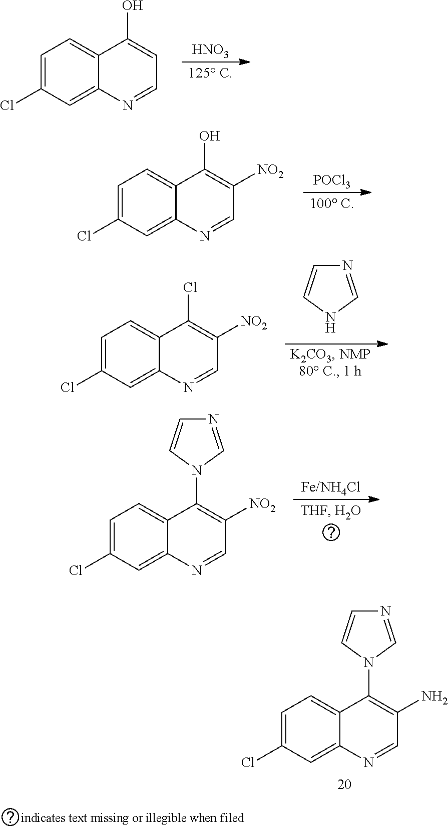

2) Preparation of Compound 15 and 21

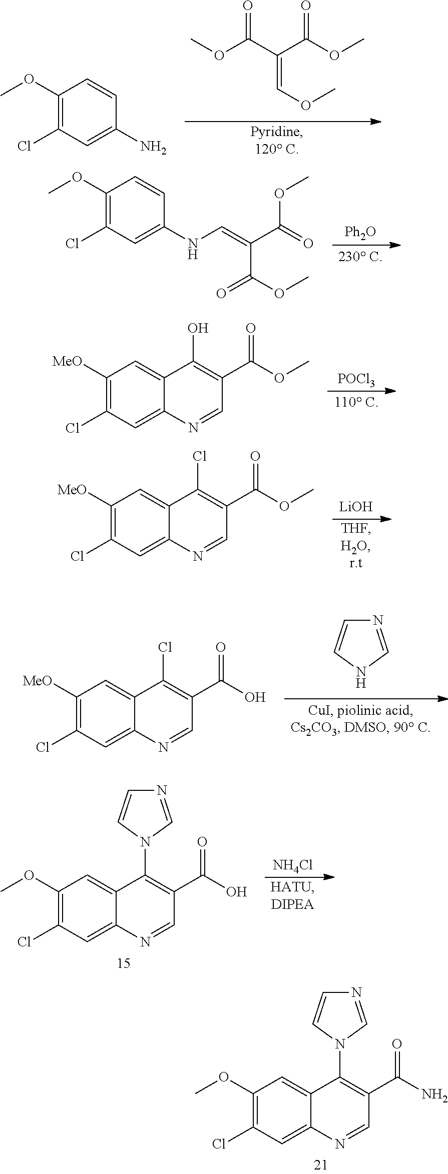

### Example 2. Compounds 1-23

The following compounds were prepared substantially according to the procedures described above and procedures familiar to the person of ordinary skill in the art:

| Cmpd. | Structure | Chemical Name | Code |
|---|---|---|---|
| 1 | 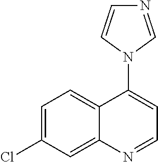 [SMILES: Clc1ccc2c(-n3ccnc3)ccnc2c1] | 7-chloro-4-(1H-imidazol-1-yl)quinolone | BBL- 0050101 |
| 2 | 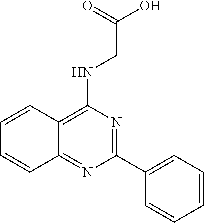 [SMILES: O=C(O)CNc1nc(-c2ccccc2)nc2ccccc12] | (2-phenylquinazolin-4-yl)glycine | BBL- 0100121 |
| 3 | 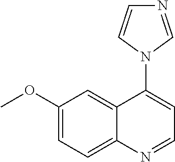 [SMILES: COc1ccc2nccc(-n3ccnc3)c2c1] | 4-(1H-imidazol-1-yl)-6-methoxyquinoline | BBL- 0100199 |
| 4 | 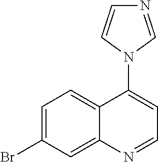 [SMILES: Brc1ccc2c(-n3ccnc3)ccnc2c1] | 7-bromo-4-(1H-imidazol-1-yl)quinoline | BBL- 0100202 |
| 5 | 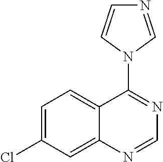 [SMILES: Clc1ccc2c(-n3ccnc3)ncnc2c1] | 7-chloro-4-(1H-imidazol-1-yl)quinazoline | BBL- 0050101 |
| 6 | 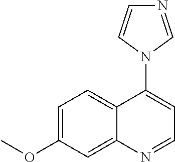 [SMILES: COc1ccc2c(-n3ccnc3)ccnc2c1] | 4-(1H-imidazol-1-yl)-7-methoxyquinoline | BBL- 0100212 |
| 7 | 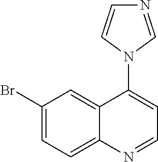 [SMILES: Brc1ccc2nccc(-n3ccnc3)c2c1] | 6-bromo-4-(1H-imidazol-1-yl)quinoline | BBL- 0100215 |
| 8 | 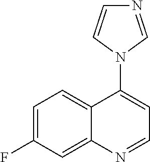 [SMILES: Fc1ccc2c(-n3ccnc3)ccnc2c1] | 7-fluoro-4-(1H-imidazol-1-yl)quinoline | BBL- 0100220 |
| 9 | 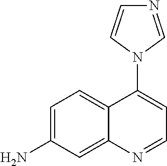 [SMILES: Nc1ccc2c(-n3ccnc3)ccnc2c1] | 4-(1H-imidazol-1-yl)quinolin-7-amine | BBL- 0100228 |
| 10 | 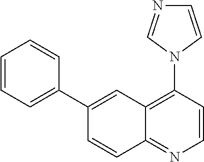 [SMILES: c1ccc(-c2ccc3nccc(-n4ccnc4)c3c2)cc1] | 4-(1H-imidazol-1-yl)-6-phenylquinoline | BBL- 0100247 |
| 11 | 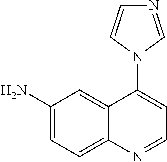 [SMILES: Nc1ccc2nccc(-n3ccnc3)c2c1] | 4-(1H-imidazol-1-yl)quinolin-6-amine | BBL- 0100252 |
| 12 | 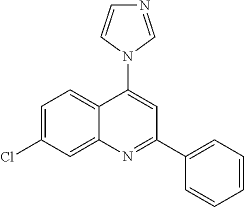 [SMILES: Clc1ccc2c(-n3ccnc3)cc(-c3ccccc3)nc2c1] | 7-chloro-4-(1H-imidazol-1-yl)-2- phenylquinoline | BBL- 0100256 |
| 13 | 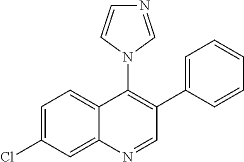 [SMILES: Clc1ccc2c(-n3ccnc3)c(-c3ccccc3)cnc2c1] | 7-chloro-4-(1H-imidazol-1-yl)-3- phenylquinoline | BBL- 0100258 |
| 14 | 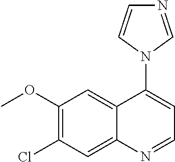 [SMILES: COc1cc2c(-n3ccnc3)ccnc2cc1Cl] | 7-chloro-4-(1H-imidazol-1-yl)-6- methoxyquinoline ¹H NMR (400 MHz, DMSO) δ 8.90 (d, J = 4.7 Hz, 1H), 8.28 (s, 1H), 8.25-8.22 (m, 1H), 7.84-7.81 (m, 1H), 7.62 (d, J = 4.7 Hz, 1H), 7.30-7.27 (m, 1H), 7.22 (s, 1H), 3.94 (s, 3H) | BBL- 0100260 |
| 15 | 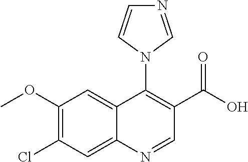 [SMILES: COc1cc2c(-n3ccnc3)c(C(=O)O)cnc2cc1Cl] | 7-chloro-4-(1H-imidazol-1-yl)-6- methoxyquinoline-3-carboxylic acid ¹H NMR (400 MHz, DMSO) δ 9.19 (s, 1H), 8.34 (s, 1H), 7.98 (s, 1H), 7.66-7.49 (m, 1H), 7.32-7.14 (m, 1H), 6.76 (s, 1H), 3.83 (s, 3H) | BBL- 0100261 |
| 16 | 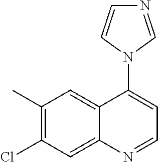 [SMILES: Cc1cc2c(-n3ccnc3)ccnc2cc1Cl] | 7-chloro-4-(1H-imidazol-1-yl)-6- methylquinoline | BBL- 0100266 |
| 17 | 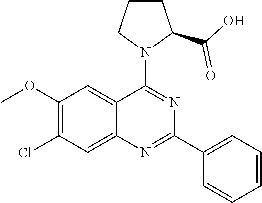 [SMILES: COc1cc2c(N3CCC[C@H]3C(=O)O)nc(-c3ccccc3)nc2cc1Cl] | (7-chloro-6-methoxy-2-phenylquinazolin- 4-yl)-L-proline | BBL- 0100261 |
| 18 | 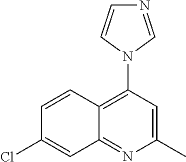 [SMILES: Cc1cc(-n2ccnc2)c2ccc(Cl)cc2n1] | 7-chloro-4-(1H-imidazol-1-yl)-2- methylquinoline | BBL- 0100259 |
| 19 | 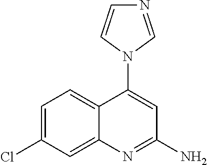 [SMILES: Nc1cc(-n2ccnc2)c2ccc(Cl)cc2n1] | 7-chloro-4-(1H-imidazol-1-yl)quinolin-2- amine ¹H NMR (400 MHz, DMSO) δ 8.07 (s, 1H), 7.65-7.61 (m, 1H), 7.59-7.54 (m, 1H), 7.38 (d, J = 8.8 Hz, 1H), 7.25-7.19 (m, 2H), 6.94 (s, 2H), 6.79 (s, 1H). | BBL- 0100309 |
| 20 | 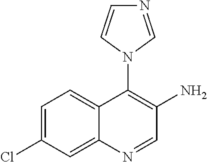 [SMILES: Nc1cnc2cc(Cl)ccc2c1-n1ccnc1] | 7-chloro-4-(1H-imidazol-1-yl)quinolin-3- amine | |
| 21 | 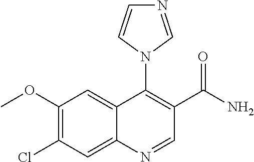 [SMILES: COc1cc2c(-n3ccnc3)c(C(N)=O)cnc2cc1Cl] | 7-chloro-4-(1H-imidazol-1-yl)-6- methoxyquinoline-3-carboxamide | |
| 22 | 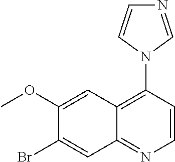 [SMILES: COc1cc2c(-n3ccnc3)ccnc2cc1Br] | 7-bromo-4-(1H-imidazol-1-yl)-6- methoxyquinoline | BBL- 0100434 |
| 23 | 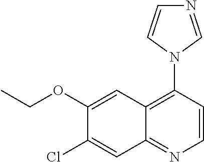 [SMILES: CCOc1cc2c(-n3ccnc3)ccnc2cc1Cl] | 7-chloro-6-ethoxy-4-(1H-imidazol-1- yl)quinolone | BBL- 0100431 |

### Example 3. Compounds 24-33

The following compounds are prepared substantially according to the procedures described above and procedures familiar to the person of ordinary skill in the art:

| Cmpd. | Structure | Chemical Name | Code |
|---|---|---|---|
| 24 | 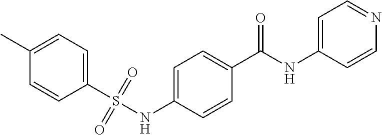 [SMILES: Cc1ccc(S(=O)(=O)Nc2ccc(C(=O)Nc3ccncc3)cc2)cc1] | 4-((4-methylphenyl)sulfonamido)-N- (pyridin-4-yl)benzamide | BBL- 0018306 |
| 25 | 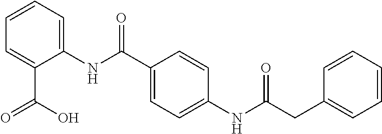 [SMILES: O=C(Cc1ccccc1)Nc1ccc(C(=O)Nc2ccccc2C(=O)O)cc1] | 2-(4-(2- phenylacetamido)benzamido)benzoic acid | BBL- 0018996 |
| 26 | 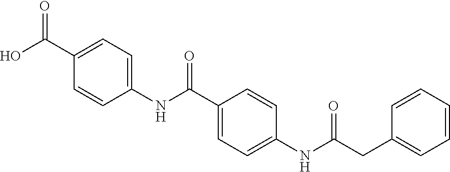 [SMILES: O=C(Cc1ccccc1)Nc1ccc(C(=O)Nc2ccc(C(=O)O)cc2)cc1] | 4-(4-(2- phenylacetamido)benzamido)benzoic acid | |
| 31 | 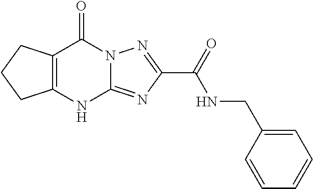 [SMILES: O=C(NCc1ccccc1)c1nc2[nH]c3c(c(=O)n2n1)CCC3] | N-benzyl-8-oxo-5,6,7,8-tetrahydro-4H- cyclopenta[d][1,2,4]triazolo[1,5- a]pyrimidine-2-carboxamide | BBL- 0031169 |
| 27 | 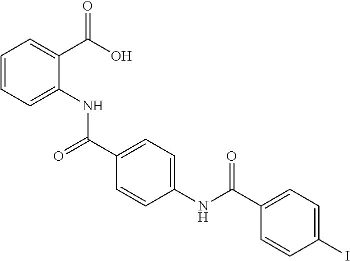 [SMILES: O=C(Nc1ccc(C(=O)Nc2ccccc2C(=O)O)cc1)c1ccc(I)cc1] | 2-(4-(4- iodobenzamido)benzamido)benzoic acid | BBL- 0100120 |
| 28 | 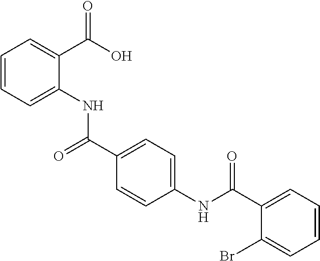 [SMILES: O=C(Nc1ccccc1C(=O)O)c1ccc(NC(=O)c2ccccc2Br)cc1] | 2-(4-(2- bromobenzamido)benzamido)benzoic acid | BBL- 0100122 |
| 32 | 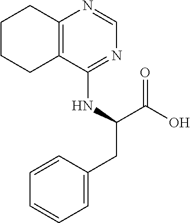 [SMILES: O=C(O)[C@@H](Cc1ccccc1)Nc1ncnc2c1CCCC2] | (5,6,7,8-tetrahydroquinazolin-4-yl)-D- phenylalanine | BBL- 0100154 |
| 29 | 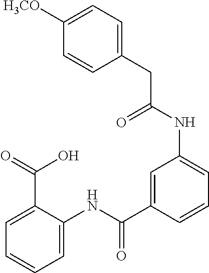 [SMILES: COc1ccc(CC(=O)Nc2cccc(C(=O)Nc3ccccc3C(=O)O)c2)cc1] | 2-(3-(2-(4- methoxyphenyl)acetamido)benzamido) benzoic acid | BBL- 0100186 |
| 30 | 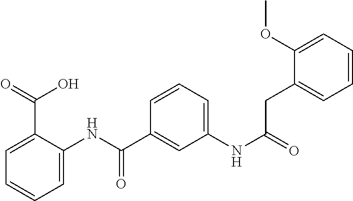 [SMILES: COc1ccccc1CC(=O)Nc1cccc(C(=O)Nc2ccccc2C(=O)O)c1] | 2-(3-(2-(2- methoxyphenyl)acetamido)benzamido) benzoic acid | BBL- 0100187 |
| 33 | 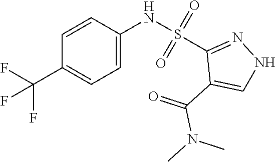 [SMILES: CN(C)C(=O)c1c[nH]nc1S(=O)(=O)Nc1ccc(C(F)(F)F)cc1] | N,N-dimethyl-3-(N-(4- (trifluoromethyl)phenyl)sulfamoyl)-1H- pyrazole-4-carboxamide | BBL- 0100198 |

### Example 4. cGAS Inhibitor

Some exemplary compounds of the disclosure were tested for inhibition of cGAS (30 nM). The results are provided in Table 1 where “A” indicates an IC₅₀ of less than 100 nM, “B” indicates an IC₅₀ of greater than 100 nM and less than 500 nM, “C” indicates an IC₅₀ of greater than 500 nM and less than 1 μM, “D” indicates an IC₅₀ of greater than 1 μM and less than 10 μM, and “E” indicates an IC₅₀ of greater than 10 μM.

TABLE 1

Inhibition

| Cmpd | cGAS | cGAS + Mn²⁺ |
|---|---|---|
| 24 | E | |
| 25 | E | E |
| 26 | E | E |
| 31 | E | |
| 1 | D | B |
| 27 | E | E |
| 2 | E | E |
| 28 | E | E |
| 32 | E | E |
| 29 | E | E |
| 30 | E | |
| 3 | E | D |
| 4 | D | B |
| 5 | E | E |
| 6 | E | D |
| 7 | E | D |
| 8 | E | E |
| 9 | | E |
| 10 | | D |
| 11 | E | D |
| 12 | D | B |
| 13 | C | B |
| 14 | C | A |
| 15 | B | A |
| 16 | D | C |
| 17 | D | E |
| 18 | C | B |
| 19 | C | |
| 22 | B | A |
| 23 | C | B |

### Example 5. cGAS Inhibitor Development

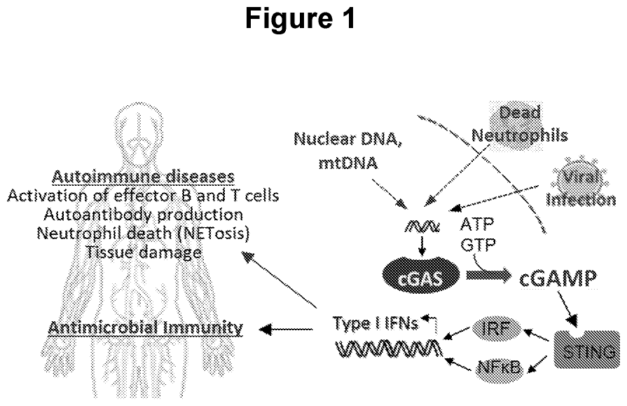

Detection of foreign nucleic acids is an important first line of defense in the immune response to microbial pathogens. However, aberrant induction of type I interferons (IFN) by self-nucleic acids causes devastating autoimmune diseases such as AGS, SLE and Sjogren's syndrome (FIG. 1). A key molecular trigger for nucleic acid-driven type I IFN induction is production of the unique cyclic dinucleotide, cGAMP, by the cytosolic DNA sensor, cGAS. The cGAS apoenzyme is enzymatically inactive; binding of non-specific dsDNA induces a transition to an active conformation that catalyzes the formation of cGAMP from ATP and GTP. cGAMP binds to the STING (stimulator of interferon genes) receptor to initiate the signaling for induction of type I IFNs. Thus the cGAS enzyme senses the primary signal for a type I IFN response and amplifies it in the form of a second messenger. Knockout studies in animal models have clearly indicated that inhibiting cGAS is a promising approach for therapeutic intervention in monogenic type I interferonopathies such as AGS and, by extension, complex diseases such as SLE.

Several novel cGAS inhibitors from a hundred thousand diversity library were discovered using a cGAS HTS, and these inhibitors had favorable structural, physicochemical and ADME/PK properties that function via distinct mechanisms. SAR-driven medicinal chemistry was used to increase the potency more than 10-fold, into the nanomolar range. The present inventors also determined that a physiological cGAS effector molecule (Mn²⁺) profoundly affects the potency of the compounds of formula (I) of the disclosure, which can inform development of cGAS drugs with more specific effects on autoimmune pathogenesis and less impact on anti-microbial immunity.

Structure-driven ligand optimization is used to advance the compounds of formula (I) of the disclosure into a mouse AGS model for efficacy testing using SAR, structural models, and molecular dynamics simulations to design and synthesize focused libraries of cGAS inhibitors with improved potency, allosteric effects, and an ADME profile suitable for a CNS drug. Structure driven ligand optimization and MOA analysis is performed for both chemotypes using human and mouse cGAS to provide compounds having an IC₅₀≤50 nM with human cGAS and 200 nM with mouse cGAS, and an IC₅₀≥500 nM off target (e.g., Kinases, GTPases, PDEs, OAS's).

Target engagement, blocking of the cGAS-STING pathway, and therapeutic efficacy in human and mouse immune cells is demonstrated by developing and/or optimizing physiologically relevant cellular assays for assessing effects of cGAS inhibitors on autoimmune disease pathways, and by demonstrating intracellular cGAS engagement and blocking of cGAS/STING-dependent inflammatory response for the compounds of formula (I) of the disclosure. Such demonstrations can include cGAS target engagement by CETSA in mouse and human cell lines, and blocking of type I IFN response and other AGS phenotypes in primary human neural and immune cells.

The presence of DNA in the cytosol of eukaryotic cells is an indicator of infection or cellular damage, and it elicits a strong immune response, driven by type I interferon (IFN) induction (FIG. 1). Though other DNA sensors have been identified in specific types of cells, the cGAS-cGAMP-STING pathway appears to be essential for DNA-mediated immune response irrespective of cell type or DNA sequence. Double strand DNA binds to a specific site on catalytically inactive cGAS monomers in a non-sequence-dependent manner. DNA binding induces formation of an activated 2:2 complex of DNA:cGAS, triggering production of a unique cyclic nucleotide G(2′-5′)pA(3′-5′)p (cGAMP) from ATP and GTP precursors. cGAMP binds to the STING protein to induce expression of type I IFNs, with autocrine and paracrine effects that lead to activation of T- and B-cells and antibody production.

Inappropriate activation of the cGAS/STING pathway contributes to the pathology of a number of autoimmune diseases (Table 2) including monogenic type I interferonopathies such as AGS and retinal vasculopathy with cerebral leukodystophysystemic (RVCL) as well as multifactorial diseases like SLE, scleroderma, and Sjögren's syndrome. These diseases cause significant pain and suffering and shorten life spans for millions of people in the U.S. alone. AGS, a rare neonatal encephalopathy that causes debilitating physical and mental impairment, results in 25% mortality in early childhood, with very few patients surviving past their teens. SLE, a far more common disease, is not usually directly fatal, but it increases mortality, most frequently from cardiovascular disease; 20% of patients die within 15 years of diagnosis. And it profoundly impacts quality of life; only 46% of working-age patients are in the workforce.

TABLE 2
Autoimmune diseases triggered by
cGAS/STING-driven IFN production.

| Disease | Effects | Prevalence in U.S. |
|---|---|---|
| SLE | 67% increased mortality | 1.5M |
| Sjogren's | Increased mortality, | 1.5M |
| Syndrome | 5% lymphoma | |
| AGS | 25% mortality by age 12 | Rare |
| RVCL | High mortality | Rare |
| | 5-10 years after onset | |

Mice studies have demonstrated that cGAS can be targeted for AGS, and by extension, for SLE. 90% of AGS patients carry mutations in one of five different DNA modifying enzymes that result in accumulation of cytoplasmic DNA, most notably the dsDNA exonuclease Trex1 (23%) or RNase H2 (53%), which removes RNA from DNA:RNA hybrids. Knocking out these nucleases and/or knocking in inactivating AGS mutations causes lethal autoimmune disease in mice. Genetic ablation of cGAS or STING in the nuclease-deficient mice protects against lethality and eliminates the key autoimmune phenotypes, including interferon stimulated gene (ISG) induction, autoantibody production, and T-cell activation. Elimination of cGAS was in mice lacking DNase II, a lysosomal endonuclease that clears DNA from dead cells, provided similar results.

Mutations that impair the function of RNAse H2, Trex1, and other nucleic acid modifying enzymes also occur with low frequency in SLE, and lupus-like inflammatory disease has been recapitulated in mice carrying the TREX1 D18N mutation that causes familial chilblain lupus. cGAS can also be targeted in idiopathic SLE. In a recent clinical study, about one third of SLE patients showed high levels of cGAS mRNA and about 15% had detectable cGAMP in peripheral blood mononuclear cells (PBMCs); significantly, cGAMP+ patients had higher disease activity compared to patients without increased cGAMP. Moreover, cGAS/STING can drive type I IFN induction in response to oxidized mitochondrial DNA in neutrophil extracellular traps (NETs), complexes of histones, DNA, and proteases that contribute to pathogenesis in SLE and other autoimmune diseases. Similar results were observed with DNA-containing membrane vesicles isolated from SLE serum.

No drugs have been approved specifically for AGS or any other monogenic type I interferonopathies. Current treatment options are limited to intravenous or oral immunosuppressors and intravenous immunoglobulins during the acute phases, with often only partial control of the flares. Similarly, SLE is treated with over-the counter anti-inflammatories, corticosteroids, and immunosupressives such as cyclophosphamide and methotrexate with serious side effects, including cancer. The only targeted therapy approved for SLE is Benlysta, a mAb against B-cell activating factor (BAFF), which reduces the risk of severe flares and allows lower doses of immunosuppressive in most patients, but is not curative.

Janus Kinase (JAK) and reverse transcriptase inhibitors (RTIs) are the first targeted therapies to reach the clinic for AGS. JAKs transduce signals from the type I IFN receptor, IFNAR1, to downstream signaling components to induce ISG expression. The use of RTIs is based on studies showing that retrotransposon cycling generates cytoplasmic DNA that triggers an IFN response in Trex1-deficient mice. Direct and indirect targeting of the self-nucleic acids that trigger type I IFN induction is also under investigation for SLE; e.g. recombinant nucleases. Several therapies are being tested in clinical trials for SLE, including mAbs that block IFNα or IFNAR1, blocking IFNAR1 signal transduction; e.g., JAK inhibitors, and targeting cell types activated by type I IFNs; e.g., B- and T-cells. However, such IFN-targeting therapies can be inefficient.

cGAS is the DNA sensor that triggers a type I IFN response in 90% of AGS patients, and could perform a similar role in a significant fraction of SLE patients. Blocking the trigger for type I IFN production could be more efficient pharmacodynamically than intervening with downstream targets in the IFNAR/JAK/STAT pathway. Because cGAS is the signal amplification step in the pathway, inhibiting cGAS could be more effective than drugs that target a specific nucleic acid population (cGAS is the common sensor for any DNA that reaches the cytoplasm, regardless of origin). Moreover, aberrant type I IFN induction is triggered by multiple sources of self-DNA, some of which could be unknown. Lastly, most of the IFN-targeting drugs in clinical development are biologics; a small molecule cGAS inhibitor could be relatively inexpensive and provide for better CNS exposure.

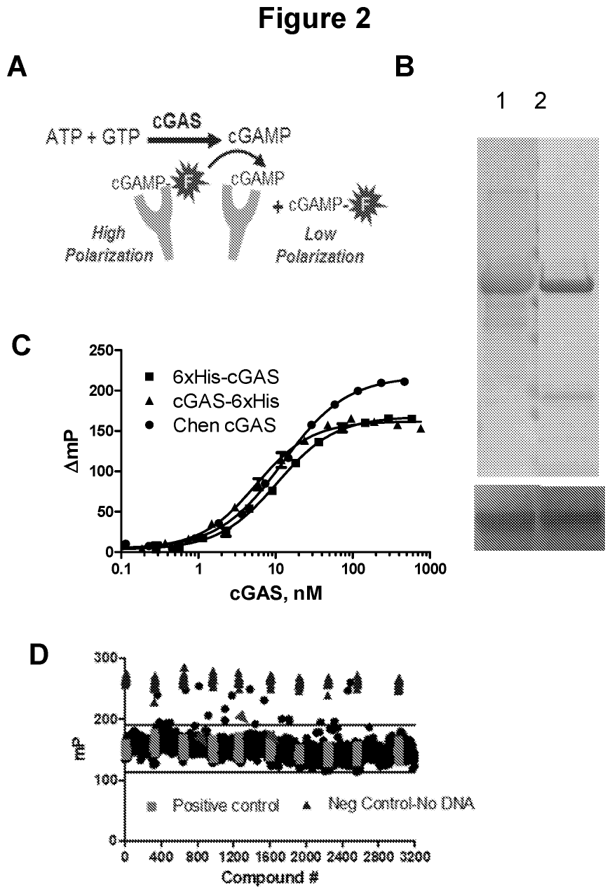

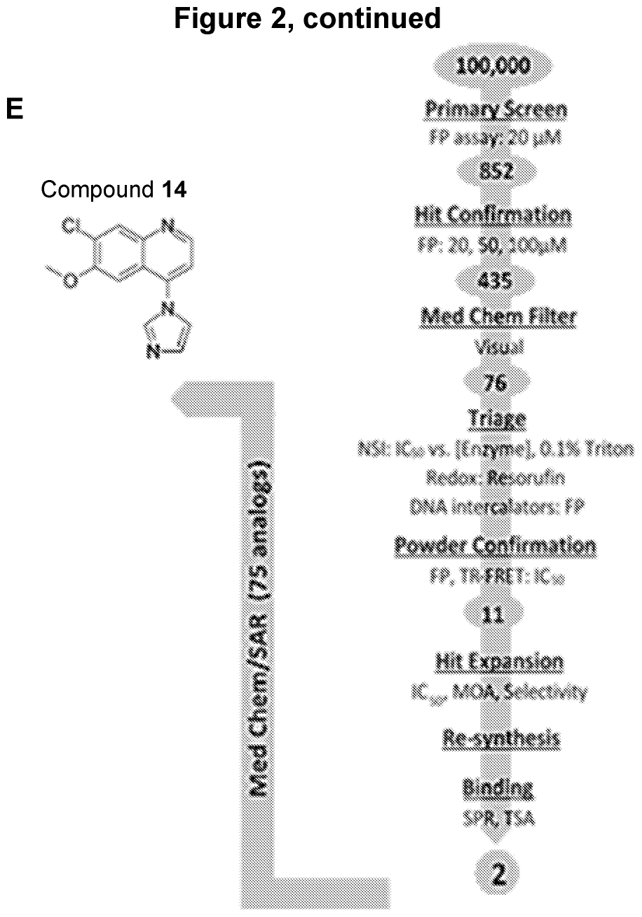

A homogenous cGAS enzymatic assay was developed with fluorescence polarization (FP) and time-resolved Forster resonance energy transfer (TR-FRET) readouts (FIG. 2A-D). The cGAS assay was used to screen 100,000 compounds with full-length human cGAS (FIG. 2E), resulting in the identification of novel chemotypes, two of which are further developed in a structure-driven hit-to-lead study (Table 3, below). The assay performance was robust, as indicated by respective Z and Z′ values of 0.59 and 0.63 in the screen; compounds with polarization values greater than three SDs from the mean were considered hits; a scatterplot from 10 plates (3,200 compounds) is shown in FIG. 2D.

TABLE 3
Compound Properties.

| | Compound | | |
|---|---|---|---|
| | 14 | | 1 |
| IC₅₀ (5 mM Mg²⁺) | 0.55 ± 0.01 | μM | 5.4 μM |
| IC₅₀ (5 mM Mg²⁺, 0.2 mM Mn²⁺) | 0.048 ± 0.002 | μM | 0.79 μM |
| IC₅₀ (Mouse cGAS) | 0.093 | μM | |
| Molecular Weight (Da) | 259.7 | | 229.7 |
| CNS MPO Score (Scale, 0-6) | 5.85 | | |
| Kₛₒₗ (PBS, pH 7.4) | 224.1 | μM | |
| Human MS Stability (NADPH) t_{1/2} | 115.9 | min | |
| Mouse MS Stability (NADPH) t_{1/2} | 21.3 | min | |

| | |
|---|---|
| MDCK-MDR Permeability A→B, | 76.7 × 10⁻⁶ cm/s, 0.5 |
| efflux ratio | |
| MOA | GTP Competitive |
| Off-Target Activity (Kinase, | None |
| PDE, GTPase, ENPP1) | |

Legend: CNS MPO: central nervous system multiparameter optimization; MS: microsomal; MDCK-MDR: Madin-Darby Canine Kidney cells-Multi Drug Resistance pump; PDE: phosphodiesterase; ENPP1: ectonucleosidase 1.

Following confirmation of hits at three concentrations and removal of compounds with visually evident reactivity or metabolic liabilities, non-stoichiometric inhibitors, aggregators, DNA intercalators and redox-active compounds were triaged using established assays. Initial SAR based on more than 100 commercially available analogs provided additional confidence that the compounds of the disclosure were bonafide inhibitors, and informed potential scaffold hops and toleration for modifications. The compounds of formula (I) of the disclosure were selected for advancement into a hit-to-lead study. Compound 1 and related analogs (i.e., compounds of formula (I)) did not bind appreciably in SPR but were found to stabilize cGAS in thermal shift assays (TSA) in the presence of ATP, GTP and dsDNA, indicating that this chemotype may bind specifically to dimerized cGAS. The compounds of formula (I) of the disclosure are competitive with GTP and its potency is enhanced in the presence of Mn²⁺.

The compounds of formula (I) of the disclosure were tested for improved potency and other drug-like properties. Certain properties of compound 14 are provided below (see also Table 3, above):
Stoichiometric binding (1:1) to cGAS, demonstrated both by enzymatic analysis and biophysical binding studies (SPR, TSA) and concordance between biochemical IC₅₀ and K_{d};
Properties including low MW (<300 Da), chemically tractability, no Lipinski violations, reactive groups, PAINS, or other structural alerts and very favorable physicochemical properties as exemplified in CNS MPO scores greater than 5 (a score greater than 4 on a scale of 0-6 is generally indicative of CNS permeability);
In vitro ADME/PK properties including metabolic stability in both mouse and human, membrane permeability and no indication of MDR-1-mediated export (which can decrease BBB permeability);
Selectivity, demonstrated though a lack of inhibitory activity at 200 μM with a panel of nucleotide-utilizing enzymes, including kinases, a GTPase, a phosphodiesterase, and a nucleotidase that metabolizes cGAMP.

The release of MnCl₂ from organelles into the cytoplasm can play a critical role in initiating a cGAS-dependent anti-viral immune response, both in cells and in mice: Mn²⁺ binding to cGAS stimulates production of cGAMP in the presence of very low concentrations of dsDNA that would otherwise be non-stimulatory. Accordingly, the effect of Mn²⁺ might on pharmacological modulation of cGAS was tested. Known human cGAS inhibitors (the antimalarial quinacrine and PF06928215) were shown to be significantly less potent when Mn²⁺ was present at a physiological concentration (200 μM), with decreases in IC₅₀ as much as 100-fold. The potency of the compounds of formula (I) of the disclosure was increased by as much as 20-fold by physiological levels of MnCl₂ (see Table 3, above). SAR including Mn²⁺ sensitivity could improve the therapeutic efficacy of a cGAS drug, while limiting its immunocompromising effects by modulating its sensitivity to Mn²⁺.

Detecting cGAMP in cell and tissue samples could provide a simple, direct way to monitor the action of lead molecules that target cGAS in animal models, and eventually for stratification and monitoring of patients in clinical studies; e.g., AGS patients or SLE patients with high levels of cGAMP in PBMCs as candidates for cGAS inhibitors. Currently, cGAMP is detected in cell lysates using a time-consuming LC-MS protocol. Therefore, the use of cGAMP as a biomarker can allow selection of patients likely to respond to a cGAMP inhibitor.

### Example 6. Structure-Based Design of cGAS Inhibitors with Improved Potency, Allosteric Effects, and an ADME Profile Suitable for a CNS Drug

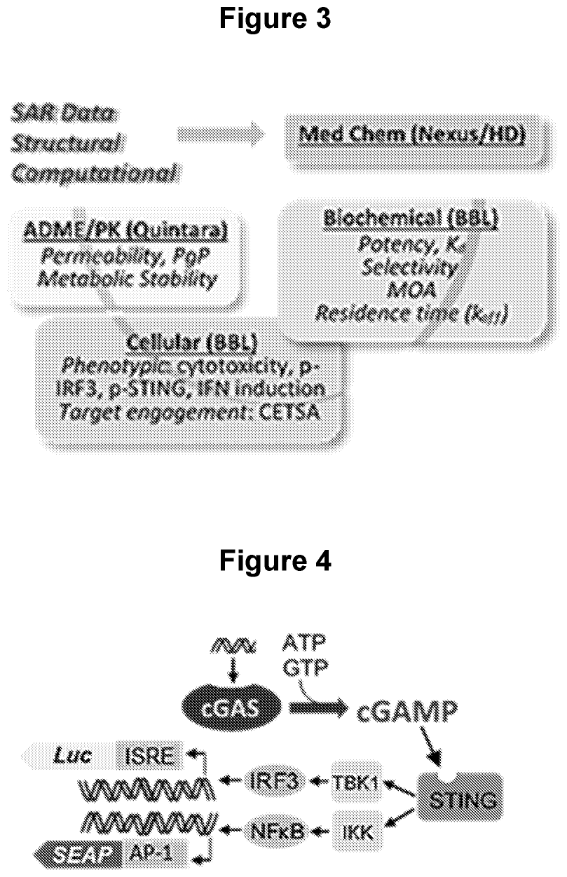

A highly efficient platform for preclinical drug discovery (FIG. 3) was assembled, providing for development of cGAS inhibitors, which is improved by the addition of a powerful computational modeling method and in vivo PK studies (FIG. 3). Compound 14 was advanced to animal studies to explore whether and how the differences in MOA and Mn²⁺ sensitivities impact therapeutic utility. Computational and SAR efforts are biased toward development of allosteric inhibitors, because allosteric drugs often have longer residence times and greater selectivity as compared with purely competitive drugs. These characteristics can allow lower and less frequent dosing, which could help prevent adverse effects from systemic immune system inhibition. Moreover, binding of dsDNA to cGAS induces a conformational transition in an activation loop, not unlike the displacement of inhibitory domains by autophosphorylation in protein kinases. Accordingly, inhibitors that lock the enzyme in an inactive conformation, similarly to imatinib with BCR-ABL kinase, could be developed.

For the compounds of formula (I) of the disclosure, TSA data suggest that it binds specifically to cGAS dimers, which is consistent with the positive effect of Mn²⁺ on potency, as GAS activation/dimerization is stimulated by Mn²⁺. This property can be advantageous from a pharmacodynamic perspective, because cGAS is likely to be largely in the activated, dimerized form in autoimmune patients due to the presence of cytosolic DNA.

Computational Methods:

Site identification by ligand competitive saturation (SILOS) methodology is used to probe the cGAS active site for pockets that can be exploited to create high-affinity allosteric inhibitors. SILOS combines computational functional group mapping with all-atom, explicit water MD simulations of the protein target to explore the conformational space and chemical space simultaneously. The resulting ‘FragMaps’ can reveal inducible pockets that are not evident from analysis of crystallographic structures and thus inform the design of ligands with allosteric properties. For example, the SILOS approach has identified allosteric binding sites on ERK kinase and heme oxygenase. In addition, the approach has been shown to be of utility for ligand design and development targeting a variety of proteins including, Mcl-1/Bcl-xl, Bcl-6, the β2-adrenergic receptor and mGluR5 among others.

Biochemical and Biophysical Analysis:

Potency and MOA studies, including Mn²⁺ sensitivity, are performed using the cGAS enzymatic assay. Dose response experiments are used to determine IC₅₀ values under basal conditions (5 mM MgCl₂, 100 μM ATP/GTP), and with the addition of physiological levels of Mn (0.2 mM) using human and mouse cGAS. Ligand optimization is driven by potency with the human enzyme; potency with mouse cGAS informs selection of an appropriate disease model for efficacy studies. Competition with ATP and GTP is assessed by comparing basal IC₅₀ values to those in the presence of saturating ATP or GTP, and subsequently confirmed by measuring velocity vs. substrate at varying ATP or GTP levels. Inhibitor residence times (1/k_{off}) are used as a key parameter for prioritizing compounds and driving SAR, because a longer residence time often results from an allosteric mechanism, and can also correlate with improved cellular activity. The cGAS enzymatic assay is used with the jump dilution method to measure residence times (inhibitor dissociation rates), as described for kinases using the very similar ADP assay. Biophysical methods, including SPR and TSAn, are used as orthologous methods for residence time measurements and k_{d} estimates.

Selectivity Profiling:

A panel of nucleotide-utilizing enzymes that included kinases (Abl1, PKA, TBK1—which transduces cGAS/STING signals, see FIG. 4) a GTPase (Rac1), a phosphodiesterase (PDE4A), and ENPP1, a nucleotidase that degrades cGAMP, was used preliminarily. cGAS assays were used to perform dose response measurements with cGAS inhibitors. In addition to these enzymes, inhibitors are tested with three other members of the oligoadenylate synthases (OAS), nucleic acid sensors that activate innate immunity via production of short, 2′-5′ oligoadenylate second messengers. Methods for expression and purification of the human and/or porcine enzymes in E. coli or baculovirus-infected insect cells have been developed as well as a simple, absorbance-based assay using commercially available pyrophosphate kit. In addition, an FP-based assay (competitive displacement of a fluor-cGAMP tracer) is developed to test compounds as ligands for STING.

ADME/PK:

Compounds are tested in Caco-2 and MDR1-MDCK permeability assays to provide a measure of intestinal absorption, blood-brain-permeability and efflux by P-glycoprotein (P-gp), a frequent obstacle to effective CNS delivery. CNS drugs are associated with high passive membrane permeability (Pₐₚₚ>1×10⁻⁶ cm/sec) and have low efflux ratios (Pₐₚₚ(B−A)/Pₐₚₚ(A−B)<2.5). Metabolic stability is tested using mouse and human liver microsomes incubated with NADPH for CYP-dependent metabolism and with UDPGA for glucuronidation. Compounds are tested for pharmacokinetics and brain penetration in mice using oral, intravenous and intraperitoneal administration.

### Example 7. Cellular Activity of Several Compounds of Disclosure

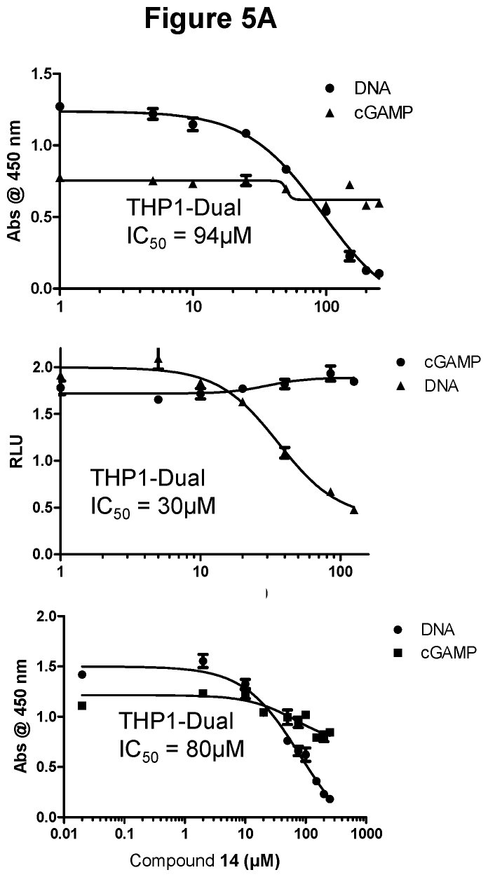

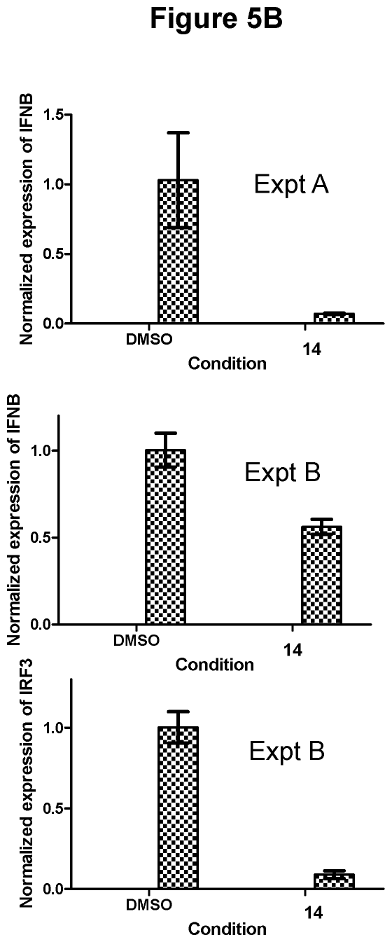

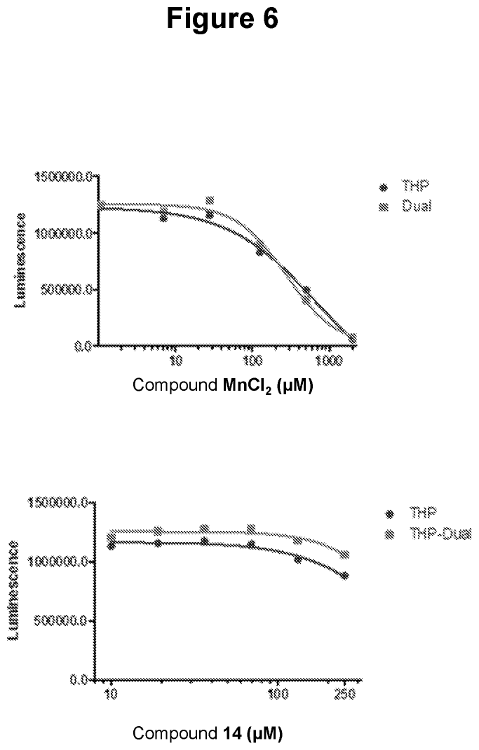

Cellular activity of several compounds of formula (I) was assessed in biochemical assays. Compound 14 was tested for effects on ISG mRNA expression, and results are shown in FIGS. 5A and 5B. For example, FIG. 5A illustrates activity of IFNβ expression, and FIG. 5B illustrates the ISG mRNA expression of compound 14 in THP1-dual cells. Finally, compound 14 was also tested for cytotoxicity and the results are shown in FIG. 6.

In summary, compound 14 shows specific inhibition of DNA-stimulated IFNβ expression as measured by ELISA as well as inhibition of reporter gene assay (not shown) and ISG expression. The results are summarized in Table 4.

TABLE 4

| Assay | | Compd. 14 (μM) |
|---|---|---|
| Enzymatic | FP Std (100 μM ATP/GTP) | 0.391 |
| | FP Physiological (1 mM ATP/GTP) | 1.92 |
| Cellular | IFNβ ELISA | 68 |
| | IRF3-Luc | 75 |
| | NFκB-SEAP | 59 |
| | ISG expression (fold decrease @ 200 μM) | 2-10× |

Some embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Various exemplary embodiments of the disclosure include, but are not limited to the enumerated embodiments listed below, which can be combined in any number and in any combination that is not technically or logically inconsistent.

Embodiment 1 provides a compound according to Formula (I):

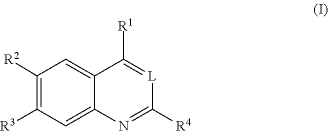

optionally in the form of a pharmaceutically acceptable salt, N-oxide, and/or a solvate or hydrate thereof, wherein L, R¹, R², R³, and R⁴ are as described above.

Embodiment 2 provides the compound of embodiment 1, wherein L is —N—.

Embodiment 3 provides the compound of embodiment 1, wherein L is —CR⁵—.

Embodiment 4 provides the compound of embodiment 3, wherein R⁵ is hydrogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —C(O)OR^{1C}, —C(O)NR^{1C}R^{1D}, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A}.

Embodiment 5 provides the compound of embodiment 3, wherein R⁵ is hydrogen, C₁-C₆ alkyl, —C(O)OR^{1C}, aryl optionally substituted with one or more R^{1B}, or heteroaryl optionally substituted with one or more R^{1B}.

Embodiment 6 provides the compound of embodiment 3, wherein R⁵ is hydrogen, phenyl, or —C(O)OH.

Embodiment 7 provides the compound of embodiment 3, wherein R⁵ is hydrogen.

Embodiment 8 provides the compound of any of embodiments 1-7, wherein R¹ is heteroaryl optionally substituted with one or more R^{1B}.

Embodiment 9 provides the compound of any of embodiments 1-7, wherein R¹ is imidazol-1-yl optionally substituted with one or more R^{1B}.

Embodiment 10 provides the compound of any of embodiments 1-7, wherein R¹ is unsubstituted imidazol-1-yl.

Embodiment 11 provides the compound of any of embodiments 1-10, wherein R² is hydrogen, halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or phenyl.

Embodiment 12 provides the compound of any of embodiments 1-10, wherein R² is hydrogen, halogen, C₁-C₆ alkyl, —NH₂, C₁-C₆ alkoxy, or phenyl.

Embodiment 13 provides the compound of any of embodiments 1-10, wherein R² is hydrogen, methyl, or methoxy.

Embodiment 14 provides the compound of any of embodiments 1-10, wherein R² is hydrogen.

Embodiment 15 provides the compound of any of embodiments 1-14, wherein R³ is hydrogen, halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy.

Embodiment 16 provides the compound of any of embodiments 1-14, wherein R³ is hydrogen, halogen, —NH₂, or methoxy.

Embodiment 17 provides the compound of any of embodiments 1-14, wherein R³ is hydrogen, —F, —Cl, or —Br.

Embodiment 18 provides the compound of any of embodiments 1-14, wherein R³ is —Cl.

Embodiment 19 provides the compound of any of embodiments 1-18, wherein R⁴ is hydrogen, C₁-C₆ alkyl, —NH₂, —NH(C₁-C₆ alkyl), —N(C₁-C₆ alkyl)₂, aryl optionally substituted with one or more R^{1B}, or heteroaryl optionally substituted with one or more R^{1B}.

Embodiment 20 provides the compound of any of embodiments 1-18, wherein R⁴ is hydrogen, methyl, —NH₂, or phenyl.

Embodiment 21 provides the compound of any of embodiments 1-18, wherein R⁴ is hydrogen, methyl, or phenyl.

Embodiment 22 provides the compound of any of embodiments 1-18, wherein R⁴ is hydrogen.

Embodiment 23 provides the compound of embodiment 1, wherein R¹ is unsubstituted imidazol-1-yl; R³ is —Cl; and R⁴ is hydrogen, methyl, —NH₂, or phenyl.

Embodiment 24 provides the compound of embodiment 1, wherein R¹ is unsubstituted imidazol-1-yl; R³ is —Cl; and R⁴ is hydrogen.

Embodiment 25 provides the compound of embodiment 1, which is any one of compounds of Example 2, or a pharmaceutically acceptable salt, N-oxide, and/or a solvate or hydrate thereof.

Embodiment 26 provides a compound according to Formula (II):

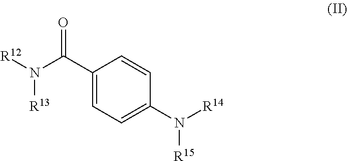

optionally in the form of a pharmaceutically acceptable salt, N-oxide, and/or a solvate or hydrate thereof, wherein R¹², R¹³, R¹⁴, and R¹⁵ are as provided above.

Embodiment 27 provides the compound of embodiment 26, wherein R¹² is heteroaryl (e.g., unsubstituted heteroaryl).

Embodiment 28 provides the compound of embodiment 26, wherein R¹² is aryl substituted with 1 R^{12A} (e.g., in which R^{12A} is —C(O)OR^{12B}).

Embodiment 29 provides the compound of any of embodiments 26-28, wherein R¹⁵ is —SO₂R^{15A} in which R^{15A} is aryl substituted with 1-2 R^{15B} (e.g., in which R^{15B} is unsubstituted C₁-C₈ alkyl).

Embodiment 30 provides the compound of any of embodiments 26-28, wherein R¹⁵ is —C(O)R^{15A} in which R^{15A} is C₁-C₈ alkyl (e.g., C₁-C₄ alkyl) substituted with 1-2 R^{15B}.

Embodiment 31 provides the compound of embodiment 30, wherein R^{15B} is unsubstituted aryl or aryl substituted with 1-2 R^{15C} (e.g., in which R^{15C} is C₁-C₄ alkoxy).

Embodiment 32 provides the compound of any of embodiments 26-28, wherein R¹⁵ is —C(O)R^{15A} in which R^{15A} is aryl substituted with 1-2 R^{15B} (e.g., in which R^{15B} is halogen).

Embodiment 33 provides the compound of embodiment 26, which is any one of compounds of Example 3, or a pharmaceutically acceptable salt, N-oxide, and/or a solvate or hydrate thereof.

Embodiment 34 provides a compound that is

N-benzyl-8-oxo-5,6,7,8-tetrahydro-4H-cyclopenta[d][1,2,4]triazolo[1,5-a]pyrimidine-2-carboxamide; (5,6,7,8-tetrahydroquinazolin-4-yl)-D-phenylalanine; N,N-dimethyl-3-(N-(4-(trifluoromethyl)phenyl)sulfamoyl)-1H-pyrazole-4-carboxamide; or a pharmaceutically acceptable salt, N-oxide, and/or a solvate or hydrate thereof.

Embodiment 35 provides the compound of any of embodiments 1-34, wherein the compound is in the form of an N-oxide.

Embodiment 36 provides the compound of any of embodiments 1-35, wherein the compound is in the form of a pharmaceutically acceptable salt.

Embodiment 37 provides the compound of any of embodiments 1-36, wherein the compound is in the form of the base compound.

Embodiment 38 provides the compound of any of embodiments 1-37, wherein the compound is in the form of solvate or hydrate.

Embodiment 39 provides the compound of any of embodiments 1-38, wherein the compound has an improved inhibition of cGAS activation in presence of Mn²⁺ compared to activation in absence of Mn²⁺ (e.g., having an IC₅₀ in the presence of Mn²⁺ that is at least 5-fold less than the IC₅₀ of the compound in otherwise identical conditions but lacking Mn²⁺).

Embodiment 40 provides a pharmaceutical composition comprising a compound according to any one of embodiments 1-39 and a pharmaceutically acceptable carrier, solvent, adjuvant or diluent.

Embodiment 41 provides a method for treating or preventing inappropriate activation of a type I interferon (IFN) response in a subject in need thereof, the method comprising administering to a subject in need of such treatment an effective amount of one or more compounds according to any one of embodiments 1-39 or a pharmaceutical composition according to embodiment 40.

Embodiment 42 provides a method of treating an autoimmune disorder, the method comprising administering to a subject in need of such treatment an effective amount of one or more compounds according to any one of embodiments 1-39 or a pharmaceutical composition according to embodiment 40.

Embodiment 43 provides the method of embodiment 42, wherein the autoimmune disorder is Aicardi-Goutieres Syndrome, retinal vasculopathy with cerebral leukodystropy, lupus erythematosus, scleroderma, or Sjogren's syndrome.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A compound according to Formula (I): 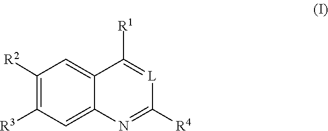
optionally in the form of a pharmaceutically acceptable salt, N-oxide, and/or a solvate or hydrate thereof, wherein:
L is —N— or —CR⁵—;
R⁵ is hydrogen, halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R^{1C}, —C(O)OR^{1C}, —C(O)NR^{1C}R^{1D}, —S(O)₀₋₂—R^{1C}, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A};
R¹ is aryl optionally substituted with one or more R^{1B}, heteroaryl optionally substituted with one or more R^{1B}, heterocycloalkyl optionally substituted with one or more R^{1A}, C₄-C₈ cycloalkyl optionally substituted with one or more R^{1A}, or —NH(C₁-C₆ alkyl) optionally substituted with one or more R^{1A};
R² is hydrogen, halogen, —NO₂, —CN, 1-C₆ alkyl, 1-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A};
R³ is hydrogen, halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, or C₁-C₆ haloalkoxy; and
R⁴ is hydrogen, halogen, —NO₂, —CN, C₁-C₆ alkyl, C₂-C₈ alkenyl, C₂-C₈ alkynyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R^{1C}, —C(O)OR^{1C}, —C(O)NR^{1C}R^{1D}, —S(O)₀₋₂—R^{1C}, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A};
wherein
each R^{1A} is independently selected from the group consisting of oxo, halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R^{1C}, —C(O)OR^{1C}, —C(O)NR^{1C}R^{1D}, and —S(O)₀₋₂—R^{1C};
each R^{1B} is independently selected from the group consisting of halogen, —NO₂, —CN, C₁-C₆ alkyl, C₁-C₆ haloalkyl, —N₃, —NH₂, —NH(C₁-C₆ alkyl), —N(C₁-C₆ alkyl)₂, —OH, C₁-C₆ alkoxy, C₁-C₆ haloalkoxy, —C(O)R^{1C}, —C(O)OR^{1C}, —C(O)NR^{1C}R^{1D}, and —S(O)₀₋₂—R^{1C};
each R^{1C} is independently selected from the group consisting of hydrogen, C₁-C₆ alkyl, aryl(C₀-C₄ alkyl), heteroaryl(C₀-C₄ alkyl), heterocyclyl(C₀-C₄ alkyl), and cyclyl(C₀-C₄ alkyl); and
each R^{1D} is independently hydrogen or C₁-C₆ alkyl.

2. The compound of claim 1, wherein L is —N—.

3. The compound of claim 1, wherein L is —CR⁵—.

4. The compound of claim 3, wherein R⁵ is hydrogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —C(O)OR^{1C}, —C(O)NR^{1C}R^{1D}, aryl optionally substituted with one or more R^{1B}, heteroaryl optionally substituted with one or more R^{1B}, heterocycloalkyl optionally substituted with one or more R^{1A}, or C₄-C₈ cycloalkyl optionally substituted with one or more R^{1A}.

5. The compound of claim 3, wherein R⁵ is hydrogen, C₁-C₆ alkyl, —C(O)OR^{1C}, aryl optionally substituted with one or more R^{1B}, or heteroaryl optionally substituted with one or more R^{1B}.

6. The compound of claim 3, wherein R⁵ is hydrogen, phenyl, or —C(O)OH.

7. The compound of claim 3, wherein R⁵ is hydrogen.

8. The compound of any of claims 1-7, wherein R¹ is heteroaryl optionally substituted with one or more R^{1B}.

9. The compound of any of claims 1-7, wherein R¹ is imidazol-1-yl optionally substituted with one or more R^{1B}.

10. The compound of any of claims 1-7, wherein R¹ is unsubstituted imidazol-1-yl.

11. The compound of any of claims 1-10, wherein R² is hydrogen, halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, or phenyl.

12. The compound of any of claims 1-10, wherein R² is hydrogen, halogen, C₁-C₆ alkyl, —NH₂, C₁-C₆ alkoxy, or phenyl.

13. The compound of any of claims 1-10, wherein R² is hydrogen, methyl, or methoxy.

14. The compound of any of claims 1-10, wherein R² is hydrogen.

15. The compound of any of claims 1-14, wherein R³ is hydrogen, halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, or C₁-C₆ haloalkoxy.

16. The compound of any of claims 1-14, wherein R³ is hydrogen, halogen, —NH₂, or methoxy.

17. The compound of any of claims 1-14, wherein R³ is hydrogen, —F, —Cl, or —Br.

18. The compound of any of claims 1-14, wherein R³ is —Cl.

19. The compound of any of claims 1-18, wherein R⁴ is hydrogen, C₁-C₆ alkyl, —NH₂, —NH(C₁-C₆ alkyl), —N(C₁-C₆ alkyl)₂, aryl optionally substituted with one or more R^{1B}, or heteroaryl optionally substituted with one or more R^{1B}.

20. The compound of any of claims 1-18, wherein R⁴ is hydrogen, methyl, —NH₂, or phenyl.

21. The compound of any of claims 1-18, wherein R⁴ is hydrogen, methyl, or phenyl.

22. The compound of any of claims 1-18, wherein R⁴ is hydrogen.

23. The compound of claim 1, wherein R¹ is unsubstituted imidazol-1-yl; R³ is —Cl; and R⁴ is hydrogen, methyl, —NH₂, or phenyl.

24. The compound of claim 1, wherein R¹ is unsubstituted imidazol-1-yl; R³ is —Cl; and R⁴ is hydrogen.

25. The compound of claim 1, which is
7-chloro-4-(1H-imidazol-1-yl)quinolone;
(2-phenylquinazolin-4-yl)glycine;
4-(1H-imidazol-1-yl)-6-methoxyquinoline;
7-bromo-4-(1H-imidazol-1-yl)quinolone;
7-chloro-4-(1H-imidazol-1-yl)quinazoline;
4-(1H-imidazol-1-yl)-7-methoxyquinoline;
6-bromo-4-(1H-imidazol-1-yl)quinolone;
7-fluoro-4-(1H-imidazol-1-yl)quinolone;
4-(1H-imidazol-1-yl)quinolin-7-amine;
4-(1H-imidazol-1-yl)-6-phenylquinoline;
4-(1H-imidazol-1-yl)quinolin-6-amine;
7-chloro-4-(1H-imidazol-1-yl)-2-phenylquinoline;
7-chloro-4-(1H-imidazol-1-yl)-3-phenylquinoline;
7-chloro-4-(1H-imidazol-1-yl)-6-methoxyquinoline;
7-chloro-4-(1H-imidazol-1-yl)-6-methoxyquinoline-3-carboxylic acid;
7-chloro-4-(1H-imidazol-1-yl)-6-methylquinoline;
(7-chloro-6-methoxy-2-phenylquinazolin-4-yl)-L-proline;
7-chloro-4-(1H-imidazol-1-yl)-2-methylquinoline;
7-chloro-4-(1H-imidazol-1-yl)quinolin-2-amine;
7-chloro-4-(1H-imidazol-1-yl)quinolin-3-amine;
7-chloro-4-(1H-imidazol-1-yl)-6-methoxyquinoline-3-carboxamide;
7-bromo-4-(1H-imidazol-1-yl)-6-methoxyquinoline;
7-chloro-6-ethoxy-4-(1H-imidazol-1-yl)quinolone;
or a pharmaceutically acceptable salt, N-oxide, and/or a solvate or hydrate thereof.

26. A compound according to Formula (II): 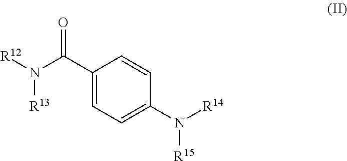
optionally in the form of a pharmaceutically acceptable salt, N-oxide, and/or a solvate or hydrate thereof, wherein:
R¹² is selected from C₁-C₈ alkyl, C₂-C₈ alkenyl and C₂-C₈ alkynyl, each optionally substituted with 1-2 R^{12A}, cycloalkyl and heterocycloalkyl, each optionally substituted with 1-2 R^{12A}, aryl and heteroaryl, each optionally substituted with 1-5 R^{12A}, in which each R^{12A} is independently oxo, optionally substituted C₁-C₄ alkyl, C₁-C₄ fluoroalkyl, halogen, —CN, —SF₅, —N₃, —C(O)OR^{12B}, —SR^{12B}, —S(O)₁₋₂R^{12B}, —OR^{12B}, —(OCH₂CH₂O)ₙ—R^{12C} in which n is 1-4, —N(R^{12C})C(O)CH₂—O—(CH₂CH₂O)ₙ—R^{12C} in which n is 0-3, —C(O)NR^{12B}(CH₂CH₂O)ₙR^{12C} in which n is 0-3, —NR^{12C}R^{12B} and —C(O)R^{12B}, each R^{12B} is independently selected from H, C₁-C₃ alkyl, and —S(O)₁₋₂N(R^{12C})R^{12C}, and each R^{12C} is independently selected from H and C₁-C₃ alkyl,
R¹³ and R¹⁴ are each independently selected from H and C₁-C₃ alkyl; and
R¹⁵ is selected from —S(O)₁₋₂R^{15A} and —C(O)R^{15A};
wherein
in which R^{15A} is C₁-C₈ alkyl, C₂-C₈ alkenyl and C₂-C₈ alkynyl, each optionally substituted with 1-2 R^{15B}, cycloalkyl and heterocycloalkyl, each optionally substituted with 1-2 R^{15B}, aryl and heteroaryl, each optionally substituted with 1-5 R^{15B},
in which each R^{15B} is independently halogen, —CN, —NHR^{15C}, —OR^{15C}, C₁-C₈ alkyl, C₂-C₈ alkenyl and C₂-C₈ alkynyl, each optionally substituted with 1-2 R^{15C}, cycloalkyl and heterocycloalkyl, each optionally substituted with 1-2 R^{15C}, aryl and heteroaryl, each optionally substituted with 1-5 R^{15C},
in which each R^{15C} is independently halogen, C₁-C₄ alkyl, or C₁-C₄ alkoxy.

27. The compound of claim 26, wherein R¹² is heteroaryl (e.g., unsubstituted heteroaryl).

28. The compound of claim 26, wherein R¹² is aryl substituted with 1 R^{12A} (e.g., in which R^{12A} is —C(O)OR^{12B}).

29. The compound of any of claims 26-28, wherein R¹⁵ is —SO₂R^{15A} in which R^{15A} is aryl substituted with 1-2 R^{15B} (e.g., in which R^{15B} is unsubstituted C₁-C₈ alkyl).

30. The compound of any of claims 26-28, wherein R¹⁵ is —C(O)R^{15A} in which R^{15A} is C₁-C₈ alkyl (e.g., C₁-C₄ alkyl) substituted with 1-2 R^{15B}.

31. The compound of claim 30, wherein R^{15B} is unsubstituted aryl or aryl substituted with 1-2 R^{15C} (e.g., in which R^{15C} is C₁-C₄alkoxy).

32. The compound of any of claims 26-28, wherein R¹⁵ is —C(O)R^{15A} in which R^{15A} is aryl substituted with 1-2 R^{15B} (e.g., in which R^{15B} is halogen).

33. The compound of claim 26, which is:
4-((4-methylphenyl)sulfonamido)-N-(pyridin-4-yl)benzamide;
2-(4-(2-phenylacetamido)benzamido)benzoic acid;
2-(4-(2-phenylacetamido)benzamido)benzoic acid;
2-(4-(4-iodobenzamido)benzamido)benzoic acid;
2-(4-(2-bromobenzamido)benzamido)benzoic acid;
2-(3-(2-(4-methoxyphenyl)acetamido)benzamido)benzoic acid;
2-(3-(2-(2-methoxyphenyl)acetamido)benzamido)benzoic acid;
or a pharmaceutically acceptable salt, N-oxide, and/or a solvate or hydrate thereof.

34. A compound that is
N-benzyl-8-oxo-5,6,7,8-tetrahydro-4H-cyclopenta[d][1,2,4]triazolo[1,5-a]pyrimidine-2-carboxamide;
(5,6,7,8-tetrahydroquinazolin-4-yl)-D-phenylalanine;
N,N-dimethyl-3-(N-(4-(trifluoromethyl)phenyl)sulfamoyl)-1H-pyrazole-4-carboxamide;
or a pharmaceutically acceptable salt, N-oxide, and/or a solvate or hydrate thereof.

35. The compound of any of claims 1-34, wherein the compound is in the form of an N-oxide.

36. The compound of any of claims 1-35, wherein the compound is in the form of a pharmaceutically acceptable salt.

37. The compound of any of claims 1-36, wherein the compound is in the form of the base compound.

38. The compound of any of claims 1-37, wherein the compound is in the form of solvate or hydrate.

39. The compound of any of claims 1-38, wherein the compound has an improved inhibition of cGAS activation in presence of Mn²⁺ compared to activation in absence of Mn²⁺ (e.g., having an IC₅₀ in the presence of Mn²⁺ that is at least 5-fold less than the IC₅₀ of the compound in otherwise identical conditions but lacking Mn²⁺).

40. A pharmaceutical composition comprising a compound according to any one of claims 1-39 and a pharmaceutically acceptable carrier, solvent, adjuvant or diluent.

41. A method for treating or preventing inappropriate activation of a type I interferon (IFN) response in a subject in need thereof, the method comprising administering to a subject in need of such treatment an effective amount of one or more compounds according to any one of claims 1-39 or a pharmaceutical composition according to claim 40.

42. A method of treating an autoimmune disorder, the method comprising administering to a subject in need of such treatment an effective amount of one or more compounds according to any one of claims 1-39 or a pharmaceutical composition according to claim 40.

43. The method of claim 42, wherein the autoimmune disorder is Aicardi-Goutieres Syndrome, retinal vasculopathy with cerebral leukodystropy, lupus erythematosus, scleroderma, Sjögren's syndrome, age-related macular degeneration, pancreatitis, ischemia, inflammatory bowel disease, nonalcoholic steatohepatitis, or Parkinson's disease.
